(19) United States
(12) Patent Application Publication
(10) Pub. No.: US 2007/0078174 A1
(43) Pub. Date: Apr. 5, 2007

(54) Tizanidine compositions and methods of treatment using the compositions

(76) Inventors: Moshe Flashner-Barak, Petach Tikva (IL); Alon Gavin Judelman, Beth Shemesh (IL)

Correspondence Address: KENYON & KENYON LLP, ONE BROADWAY, NEW YORK, NY, 10004, US

(21) Appl. No.: 11/497,666

(22) Filed: Aug. 1, 2006

Related U.S. Application Data
(60) Provisional application No. 60/704,731, filed on Aug. 1, 2005.
(60) Provisional application No. 60/819,074, filed on Jul. 6, 2006.

(51) Int. Cl. A61K 31/433

(52) U.S. Cl. 514/363

(57) ABSTRACT

The invention is directed to methods of treating spasticity in patient having a neurological disease comprising administering to a patient in need of such treatment a tizanidine formulation providing a tizanidine blood concentration of at least about 900 pg/ml for about five hours, wherein the formulation is administered prior to bedtime.

### Related Applications

This application claims the benefit of U.S. Provisional Application Nos. 60/704,731 filed Aug. 1, 2005 and 60/819,074 filed Jul. 6, 2006, hereby incorporated by reference.

### BACKGROUND OF THE INVENTION

Cerebral palsy results from a non-progressive injury to the developing central nervous system and produces motor dysfunction, movement disorders, mental deficits and impaired function. Although the CNS lesion occurs once and remains constant, expression of this lesion is affected by the interactions of growth, development, maturation and disease processes that may confound the clinical picture.

Motor dysfunction associated with cerebral palsy may include spasticity, rigidity and weakness. Spasticity is a common syndrome occurring in over 80% of cerebral palsy patients. It is characterized by increased muscle tone, resistance/difficulty in extending muscles, and excessive activation of skeletal muscles (such as spasms and exaggerated tendon jerks) due to hyperexcitability of the stretch reflex. Additionally, spasticity may be accompanied by pain, weakness, fatigue and lack of dexterity. The mechanism of spasticity-related pain is not well understood, but the pain may be associated with spasticity, as well as the resulting impairment and deformity. The increase in muscle tone affects the patients' gait, posture, sleep, and ability to perform everyday activities and makes physiotherapy and nursing care of bedridden patients difficult. If excessive spasticity is untreated, it can lead to tendon contractures, deformities, pain, and significant physical impairment, which have a negative impact on health-related quality of life.

Spasticity is associated with sleep disturbance. It negatively impacts on sleep and causes arousal through the mechanisms of muscle spasm and pain. Disturbances in sleep are a common syndrome in neurological conditions. Sleep disturbances are often secondary to pain or to spasticity. Sleep disturbances lead to daytime fatigue or sleepiness and constitute a significant factor in lowering quality of life for patients with these conditions.

Muscle spasms cause uncontrolled limb movements of various intensities and pain, either acute pain directly due to the muscle spasm or sub-acute pain due to unrelieved uncomfortable posturing. This affects the underlying sleep cycle by causing 1) prolonged sleep onset, 2) shortened duration of sleep, and 3) frequent awakenings. A fundamentally altered sleep cycle has far-reaching lifestyle ramifications for the patient and the care-givers.

Treatment of spasticity may be divided into two categories: (a) rehabilitative techniques (physiotherapy), and (b) interventional therapy (operative and pharmacologic). Most adult patients are treated with physical therapy alone with little regard to medical treatments available.

Tizanidine hydrochloride is a centrally acting (alpha)₂-adrenergic agonist indicated for the treatment of spasticity. It is used to treat spasticity in general. Tizanidine hydrochloride may be use to treat particular types of spasticity, such as spasticity in multiple sclerosis, spasticity caused by spinal chord injury, and spasticity caused by stroke or brain injury. Recently, tizanidine hydrochloride has been evaluated for the treatment of chronic headache with promising results.

Tizanidine is slightly soluble in water and the solubility decreases with rising pH. The bioavailability of tizanidine is relatively variable from patient to patient as is the clinical response to plasma drug levels, necessitating titration of the dose level on an individual basis. Tizanidine is normally dosed in an immediate release oral formulation and has been dosed as in a controlled release oral formulation. When tizanidine hydrochloride is administered orally it is absorbed essentially completely with an absolute bioavailability of about 40% due to extensive hepatic first pass metabolism. Tizanidine can cause hepatic toxicity which is reason for careful control of the dose level and plasma levels. Another prevalent side effect of tizanidine is somnolence or sedation. This somnolence limits treatment of spasticity and/or muscle spasms with tizanidine because of the effect on the patient. Daytime activity is lowered by the somnolence and/or sedation or fatigue making improvements in spasticity of limited usefulness.

Case reports have suggested using tizanidine or other sedating spasticity drugs for nocturnal use to improve sleep by treating nocturnal spasms. Tizanidine's terminal half life is reported to be 2.5 hours; therefore, frequent dosing is needed and the effectiveness would be expected to wear off after a few hours. Treatment with tizanidine before sleep would be expected to treat the first half of the night only and surely not have any effect on spasticity the following morning.

We have found that treatment of patients having a neurological disease with long acting tizanidine formulations prior to sleep benefit in having beneficial sleep and better quality of life as well as providing for treatment of next morning spasticity.

### SUMMARY OF THE INVENTION

In one embodiment, the invention encompasses methods of treating spasticity in patient having a neurological disease comprising administering to a patient in need of such treatment a tizanidine formulation providing a tizanidine blood concentration of at least about 900 pg/ml for about five hours. The neurological disease may be at least one of cerebral palsy, multiple sclerosis, stroke, restless leg syndrome, spinal cord injury, or traumatic brain injury. The tizanidine formulation may be a controlled release formulation, a sublingual formulation, buccal formulation, or a high dose immediate release formulation. The controlled release formulation may be in the form of a tablet, capsule, lozenge, troche, pastille, pill, drop, gel, viscous liquid, or spray. The sublingual formulation may also be in the form of a tablet, capsule, lozenge, troche, pastille, pill, drop, gel, viscous liquid, or spray. In another embodiment, the invention encompasses a sublingual formulation having an average AUC of about 12000 h*pg/g for a 4 mg dose. In yet another embodiment, the invention encompasses a sublingual formulation having an average AUC of about 20000 h*pg/g for a 8 mg dose. The sublingual formulation releases tizanidine in less than about 20 minutes, preferably in less than about 5 minutes. The tizanidine formulation may be administered prior to bedtime.

In another embodiment, the invention encompasses methods of improving sleep or sleep quality in a patient with a neurological disease comprising administering to a patient in need of such treatment a tizanidine formulation providing a tizanidine blood concentration of at least about 900 pg/ml for about five hours.

In yet another embodiment, the invention encompasses methods of reducing daytime fatigue or sleepiness in a patient having a neurological disease comprising administering to a patient in need of such treatment a tizanidine formulation providing a tizanidine blood concentration of at least about 900 pg/ml for about five hours.

In another embodiment, the invention encompasses methods of improving daytime quality of life in a patient having a neurological disease comprising administering to a patient in need of such treatment a tizanidine formulation providing a tizanidine blood concentration of at least about 900 pg/ml for about five hours.

### BRIEF DESCRIPTION OF THE FIGURES

FIG. 1 illustrates the tizanidine blood concentration over time after administration of a sublingual dose (2 mg) as compared to the commercially available oral dose (4 mg Zanaflex®).

FIG. 2 illustrates the tizanidine blood concentration over time after administration of a sublingual dose (4 mg) as compared to the commercially available oral dose (4 mg Zanaflex®).

FIG. 3 illustrates the tizanidine blood concentration over time after administration of a sublingual dose (8 mg) as compared to the commercially available oral dose (4 mg Zanaflex®).

FIG. 4 illustrates raw actigraphy data of cerebral palsy patient L.

FIG. 5 illustrates raw actigraphy data of cerebral palsy patent R.

FIG. 6 illustrates raw actigraphy data of cerebral palsy patent A.

FIG. 7 illustrates raw actigraphy data of cerebral palsy patient E.

FIG. 8 illustrates raw actigraphy data of cerebral palsy patient M.

FIG. 9 illustrates raw actigraphy data of cerebral palsy patient Z.

FIG. 10 illustrates raw actigraphy data of cerebral palsy patient O.

FIG. 11 illustrates sleep efficiency for cerebral palsy patients A, E. L. M. R. and Z during periods on and off medication.

FIG. 12 illustrates wake minutes for cerebral palsy patients A, E. L. M. R. and Z during periods on and off medication.

FIG. 13 illustrates sleep minutes for cerebral palsy patients A, E. L. M. R. and Z during periods on and off medication.

FIG. 14 illustrates sleep efficiency analyzed by quarter of the night for cerebral palsy patients.

FIG. 15 illustrates true sleep analyzed by quarter of the night for cerebral palsy patients.

FIG. 16 illustrates sleep-wake transitions analyzed by quarter of the night for cerebral palsy patients.

FIG. 17 illustrates mean activity level analyzed by quarter of the night for cerebral palsy patients.

FIG. 18 illustrates sleep efficiency analyzed by quarter of the night for patients with multiple sclerosis.

FIG. 19 illustrates quiet sleep analyzed by quarter of the night for patients with multiple sclerosis.

FIG. 20 illustrates mean activity level analyzed by quarter of the night for patients with multiple sclerosis.

### DETAILED DESCRIPTION OF THE INVENTION

Tizanidine hydrochloride is an alpha-2 adrenergic agonist indicated for the treatment of spasticity due to multiple sclerosis or spinal cord injury. Also, tizanidine is being investigated for the treatment of lower back pain associated with paravertebral muscle spasms, chronic tension type headaches, and trigeminal neuralgia. Tizanidine is a short-acting drug requiring frequent, multiple daily dosing. Tizanidine's extensive first-pass hepatic metabolism results both in a lowered bioavailability (22-40%), as well as an increased potential for liver toxicity. Tizanidine is generally considered to be an effective anti-spastic agent, comparable to other agents, with fewer patients complaining of muscle weakness when taking the drug.

It is postulated that tizanidine positively affects sleep efficacy by modulating four different routes. (1) Sleep architecture—Sleep is broken up into cycles, where each cycle can be divided broadly into non-REM (stage1-4) and REM (stage 5) sleep. (2) Pain control-treatment of pain may often alleviate pediatric CP sleep disturbance. (3) Treatment of spasticity—treatment of spasticity is a recognized management option for the treatment of sleep disruption in this population. (4) Regulation of circadian rhythm—recent research has implicated altered circadian rhythm, following brain damage, as a cause of sleep disruption and daytime fatigue. Accordingly, tizanidine is a worthy candidate for the treatment of sleep disruption in the cerebral palsy population and in other neurological diseases.

We have discovered that treating patients who suffer from spasticity of neurological diseases with a longer acting tizanidine formulation before bedtime improves the quality of sleep and also reduces their spasticity throughout the next morning. The longer acting tizanidine formulations have the effect of lowering daytime fatigue or sleepiness and generally improving the “quality of life” parameters of the patient. The neurological diseases include, but are not limited to, at least one of cerebral palsy (CP), multiple sclerosis (MS), stroke, restless leg syndrome, spinal cord injury, or traumatic brain injury. Spasticity is measured by standardized measurement scales such as the “Ashworth scale,” and/or the “Timed Up and Go Test.” Fatigue is measured by standardized measurement scales such as the “Epworth Sleepiness Scale.” “Quality of life” is measured by quality of life questionnaires. It is posited that having a blood level of tizanidine which is effective for treating spasticity for most of the night is necessary for achieving any of the above-described effects. Preferably, the effect lasts about 5 hours or more. It is further posited that having an effective blood level of tizanidine of at least about 900 pg/ml for most of the night (about 5 hours or more) is necessary for achieving the effect. Such effects may be achieved by the administration of a controlled release formulation, a sublingual formulation, or a high dose formulation.

The sublingual formulations used in the invention have been found to have one or more of the following advantages over the conventional 2 mg or 4 mg oral tablet:

Improved bio-availability—The direct introduction of tizanidine into the systemic circulation via the sublingual route of administration was shown to increase the drug absorption and bioavailability. Consequently, the total drug dose may be reduced. The greater AUC means that a single nighttime dose will remain in the patient's therapeutic range until early morning; thus, providing extended nighttime coverage and benefit to the patient over the shorter-duration oral formulation.

Reduced inter-patient variability—The standard deviations observed for the AUC and Cₘₐₓ values demonstrated reduced inter-patient variability with a more predictable and uniform pharmacokinetic profile.

Improved side-effect profile—Studies demonstrated that drug efficacy as correlated to AUC, may be reached with a lower Cₘₐₓ. This suggests that side-effects associated with tizanidine dosing, e.g. somnolence and blood pressure, may be reduced with the sublingual formulation. Moreover, the sublingual formulation may be a much safer challenge to the hepatocytes than the high concentrations of oral drug introduced via the entero-hepatic circulation, thereby avoiding potential hepatotoxicity.

The invention encompasses a method of treating morning spasticity in a patient having a neurological disease by treating the patient in need thereof with a long acting tizanidine formulation prior to bedtime. The long acting tizanidine formulation provides a tizanidine blood concentration that is effective for treating spasticity. Typically, the long acting tizanidine formulation allows for the tizanidine concentration within the blood to be sufficient or greater than necessary for the effective treatment of spasticity for the desired time period. Generally, the treatment is administered to provide effective levels of tizanidine to allow for about five hours of sleep. One such formulation is one that provides a tizanidine blood concentration of at least about 900 pg/ml over a period of about five hours. The amount of time before bedtime that the drug may be administered will depend upon the effective duration of the tizanidine formulation. As used herein the term “effective duration” refers to the length of time that the tizanidine is at an effective blood concentration level sufficient to treat spasticity.

The invention also encompasses methods of improving sleep or improving sleep quality in a patient having a neurological disease comprising administering to a patient in need of such treatment a formulation providing a tizanidine blood concentration of at least about 900 pg/ml over a period of about five hours. As used herein, the term “improving sleep” or “improving sleep quality” means an improvement in the “Epworth sleepiness scale” or “Pittsburgh Sleep Quality Index” as determined by a clinician.

The invention also encompasses methods for reducing daytime fatigue or sleepiness in a patient having a neurological disease comprising administering to a patient in need of such treatment a formulation providing a tizanidine blood concentration of at least about 900 pg/ml over a period of about five hours. As used herein, the term “reducing daytime fatigue or sleepiness” means a statistically significant improvement in the “Paced Auditory Serial Addition Task” or the “Fatigue Severity Scale” as determined by a clinician.

The invention also encompasses methods for improving quality of life in a patient having a neurological disease comprising administering to a patient in need of such treatment a formulation providing a tizanidine blood concentration of at least about 900 pg/ml for about five hours. As used herein the term “improving quality of life” or “improved quality of life” means a statistically significant increase in the score of a quality of life questionnaire as determined by a clinician.

The invention encompasses the use of tizanidine or a pharmaceutically acceptable salt thereof for the manufacture of a medicament for: (a) treating spasticity in a patient having a neurological disease; (b) improving sleep or sleep quality in a patient with a neurological disease; (c) reducing daytime fatigue or sleepiness in a patient having a neurological disease; or (d) improving daytime quality of life in a patient having a neurological disease, wherein the medicament provides a tizanidine blood concentration of at least about 900 pg/ml over a period of about 5 hours, and the medicament is administered prior to bedtime. Preferably the medicament is a controlled release formulation, a sublingual formulation, a buccal formulation or a high dose formulation.

Also provided is the use of tizanidine or a pharmaceutically acceptable salt thereof for the manufacture of a medicament for (a) treating spasticity in a patient having a neurological disease; (b) improving sleep or sleep quality in a patient with a neurological disease; (c) reducing daytime fatigue or sleepiness in a patient having a neurological disease; or (d) improving daytime quality of life in a patient having a neurological disease, wherein the medicament is a controlled release formulation, a sublingual formulation, a buccal formulation or a high dose formulation, and the medicament is administered prior to bedtime.

In one embodiment, the tizanidine formulation provides a tizanidine blood concentration of at least about 900 pg/ml that is maintained over a period of about 5 hours after administration. Typically for an immediate release oral formulation, the tizanidine dose necessary to achieve these levels may be about at least 8 mg of tizanidine. Embodiments of the tizanidine formulation capable of achieving a tizanidine blood concentration of at least about 900 pg/ml over a period of about 5 hours after administration include, but are not limited to, controlled release formulations, zero order delivery systems, sublingual formulations, buccal formulations, or immediate release high dose formulations. As used herein, the term “high dose” when referring to a tizanidine formulation means a tizanidine dose of about 8 mg or more.

Preferably, the tizanidine formulation is administered to the patient prior to bedtime. As used herein, the term “bedtime” means the time at which an individual retires to sleep. The term “prior to bedtime” refers to the period of time before retiring to sleep. Thus, typically the term “prior to bedtime” refers to a period of time of up to about 1 hour before retiring to sleep, preferably up to about 30 minutes before retiring to sleep, and more preferably up to 15 minutes before retiring to sleep. Most preferably, the term “prior to bedtime” includes a period of time up to 5 minutes before retiring to sleep.

The high dose immediate release formulation may be in the form of a tablet, capsule, lozenge, troche, pastille, pill, drop, gel, viscous liquid, or spray. The high dose immediate release formulation contains a dose from about 8 mg to 20 mg, and preferably about 8 mg or about 16 mg of tizanidine. More preferably, the high dose immediate release formulation contains a dose of about 8 mg to about 12 mg of tizanidine.

The buccal formulation is preferably in the form of a tablet, lozenge, pastille, pill, drop, gel, viscous liquid, or spray. Preferably, the buccal formulation contains a dose of from about 2 to 20 mg, and more preferably from about 4 to 16 mg of tizanidine. Most preferably, the buccal formulation dose contains about 6 mg to about 12 mg of tizanidine. For example, the buccal formulation preferably contains a dose of about 4 mg, about 6 mg, about 8 mg, or about 12 mg of tizanidine.

The controlled release tizanidine formulations can be designed to provide tizanidine blood concentration levels of at least about 900 pg/ml over a period of about 5 hours or more after administration. The controlled release tizanidine formulation may be in the form of a tablet, capsule, lozenge, troche, pastilles, pills, drops, gels, viscous liquids, or spray. The controlled release tizanidine formulations may be designed and prepared using well known pharmaceutical principles. For example, the controlled release tizanidine formulation may include those described in U.S. publication No. 2005/118256, hereby incorporated by reference, as long as the formulation provides the tizanidine blood concentration described above.

The controlled release tizanidine formulation should be designed to have a Cₘₐₓ below about 3500 pg/ml and not have a Cₘₐₓ higher than that obtained with a 4 mg immediate release (IR) tizanidine formulation. More preferably, the controlled release tizanidine formulation has a Cₘₐₓ of at least about 900 pg/ml. The controlled release formulation preferably contains a dose of from about 2 mg to 36 mg, and more preferably from about 4 mg to 20 mg of tizanidine. Most preferably, the controlled release formulation contains a dose of about 6 mg to about 12 mg of tizanidine. For example, the controlled release formulation can contain a dose of about 4 mg, about 6 mg, about 8 mg, or about 12 mg of tizanidine. A preferred dosage form of the controlled release tizanidine formulation may be an eroding tablet for drug release made of a matrix formed from hydrogels or other polymers. Other preferred dosage forms include a capsule containing pellets. The pellets may be formulated to erode slowly to release tizanidine using polymers or hydrogels as is known in the art.

Preferred polymers used for the controlled release tizanidine formulation include, but are not limited to, at least one of hydroxypropylcellulose (HPC), hydroxypropylmethylcellulose (HPMC), polyvinylpyrrolidone (PVP), or polyethyleneoxide (PEO). The controlled release tizanidine formulation may have a controlled release coating such as Eudragit RL™, Eudragit RS™, Eudragit NE™, or other similar permeable coatings.

Another preferred dosage controlled release dosage form uses special delivery forms which are designed to give close to zero order drug delivery. Zero order drug delivery systems include, but are not limited to, an osmotic pump device such as those described in U.S. Pat. Nos. 5,817,335; 5,869,096; and 5,200,194, hereby incorporated by reference. More preferred zero order drug delivery systems are drug delivery systems such as those described in U.S. publication No. 2003/143,257 or annularly coated delivery systems such as those described in U.S. publication No. 2004/052,843, hereby incorporated by reference.

A more preferred dosage form is a tizanidine sublingual formulation. The pharmacokinetic profile using sublingual delivery has particular advantages which allow for easier titration and dose level selection. When using sublingual delivery, drug bioavailability is improved while simultaneously diminishing drug absorption variability. Furthermore, sublingual delivery allows for longer period of action by flattening out the drug delivery profile. In one embodiment, the sublingual formulation may have an average AUC of about 12000 h*pg/g for a 4 mg dose. In another embodiment, the sublingual formulation may have an average AUC of about 20000 h*pg/g for a 8 mg dose.

The sublingual formulation may be formulated into a tablet, pill, capsule, lozenge, gel, paste, drop, gel, spray, or a viscous liquid that adheres to the sublingual surface. Preferably, the sublingual formulation is in the form of a tablet, pill, drop, gel, viscous liquid, or spray. More preferably, the sublingual formulation is in the form of a tablet. Typically, the sublingual formulation contains a dose of about 2 mg to about 20 mg, preferably from about 4 mg to 16 mg, and more preferably from about 6 mg to about 12 mg of tizanidine. Preferred sublingual formulations include those containing a dose of about 2 mg, about 4 mg, about 6 mg, about 8 mg, about 12 mg, or about 16 mg of tizanidine, and more preferably about 8 mg or about 12 mg of tizanidine.

In the sublingual formulation, the tizanidine may be released during the period of time that the formulation is held under the tongue. Preferably, the tizanidine sublingual formulation releases tizanidine in less than twenty minutes. More preferably, the tizanidine sublingual formulation releases tizanidine in less than five minutes.

In a most preferred embodiment, the tizanidine sublingual formulation is formulated to protect the tizanidine containing layer both during handling and during sublingual tizanidine delivery. An inner tablet containing tizanidine is designed to disintegrate and/or dissolve quickly. An outer annular tablet affords protection of the inner tablet. The protected tizanidine formulation may be made using the methods described in U.S. publication Nos. 2003/206,954 and 2004/122,065, hereby incorporated by reference.

An especially preferred sublingual dosage form is a tablet formed by multiple compression steps into an inner tablet core containing tizanidine surrounded by an annular body. One advantage of this form is that the tizanidine-containing portion of the tablet is protected from disintegration by handling. The protected dosage form comprises a core tablet containing tizanidine sheathed in an annular body comprised of compressed powder or granular material. The core tablet has first and second opposed surfaces and a circumferential surface.

As used herein, the term “sheathing” refers to the annular body encircling the core tablet and in contact with the core tablet about its circumferential surface, leaving opposed surfaces of the core tablet substantially exposed.

In one embodiment, the core tablet containing the tizanidine is recessed in an annular body while in another it is surrounded by the annular body but not recessed within. The core tablet has opposed first and second surfaces and an outer circumferential surface extending between the opposed surfaces. The core tablet is preferably cylindrical or disk shaped for ease of manufacture. Preferably, the maximum distance across either of the opposed surfaces is from about 2 mm to about 12 mm, more preferably from about 4 mm to about 7 mm, and most preferably about 5 mm. The opposed surfaces can be flat, concave, or convex. Preferably, the opposed surfaces are flat.

The outer contour of the annular body can have any cross-section shape including, but not limited to, oval, cylindrical, elliptical, or oblong. Preferably, the cross section is cylindrically shaped. Preferably, the outer diameter of the annular body is from about 5 mm to about 15 mm, more preferably from about 7 mm to about 12 mm, and most preferably about 9 mm. The inner diameter of the annular body can be any size up to about 2 mm less than the outer diameter. Preferably, the inner diameter is 3 mm or greater.

The solid dosage form with a drug-containing core tablet sheathed in a compressed annular body of excipients can be produced using multi-compression techniques known in the art (including tooling sets) or such as those described in U.S. publication No. 2003/206,954 and PCT publication WO 03/057136, hereby incorporated by reference.

The core tablet can be formulated for any desired release profile including, but not limited to, immediate release, delayed release, burst or pulsed release, or sustained or zero order release. More preferably, the core tablet is formulated for an immediate release profile. When formulated for an immediate release profile, the core preferably contains a disintegrant like crospovidone to accelerate release. Optionally, the core tablet may contain acidulant. Other excipients used in an immediate release core tablet include a-lactose monohydrate, microcrystalline cellulose, sodium saccharine, and magnesium stearate. A preferred composition of the core tablet comprises about 1 to 10 parts tizanidine hydrochloride, about 50 to 70 parts α-lactose, about 10 to 20 parts microcrystalline cellulose, about 0.1 to 1 part sodium saccharine, and about 15 to 25 parts crospovidone, exclusive of other excipients that may be present.

The annular body can be formulated with any additional desired purpose in mind. For example, the annular body may be used for taste masking. Optionally, the annular body may contain an acidulant. The annular body can be formed of any pharmaceutically acceptable excipient. In particular, the annular body may include diluents, binders, disintegrants, glidants, lubricants, flavorants, colorants, and the like. Blending and granulation with conventional excipients is well within the knowledge of those skilled in the art of tabletting.

Preferred excipients for forming the annular body include, but are not limited to, hydroxypropyl cellulose (e.g. Klucel®), hydroxypropyl methylcellulose (e.g. Methocel®), microcrystalline cellulose (e.g., Avicel®), starch, lactose, sugars, crospovidone (e.g., Kollidon™), polyvinylpyrrolidone (e.g. Plasdone®), or calcium phosphate. Most preferred excipients for forming the annular body include α-lactose monohydrate, microcrystalline cellulose, and compressible sugar. An especially preferred ring excipient is a spray dried mixture of about 75% α-lactose monohydrate and 25% by weight of microcrystalline cellulose with a particle size distribution of d(15)<32 μm and d(90)<250 μm. One such a mixture is commercially available from Meggle AG (Wasserburg, Germany, as Microcellac™). Compressible sugar is commercially available as Nu-Tab™ (CHR. Hansen, Hnrrsholm, Denmark).

A preferred annular body composition is about 45 to 50 parts compressible sugar, about 30 to 40 parts α-lactose monohydrate, about 1 to 10 parts microcrystalline cellulose, and about 1 to 10 parts crospovidone.

While the present invention is described with respect to particular examples and preferred embodiments, it is understood that the present invention is not limited to these examples and embodiments. The present invention, as claimed, therefore includes variations from the particular examples and preferred embodiments described herein, as will be apparent to one of skill in the art.

### EXAMPLES

### Example 1

Sublingual Tablet Preparation

The sublingual tablets used in this study were formulated into an inner core of a fast disintegrating formulation containing tizanidine (2 mg) and an outer annular body of protective excipients. The inner core was made by mixing 4.5 parts tizanidine hydrochloride and 20 parts crospovidone for 2 minutes. One half part sodium saccharin, 73.6 parts of Microcellac 100™, and 0.4 parts menthol were added and mixing was continued for 3 minutes. One part magnesium stearate was added and mixing was continued for a half a minute to obtain a final mixture. The final mixture was compressed using a Manesty f3 tablet press fitted with a 5 mm flat beveled punch. The tablets formed were each of 5 mm diameter, about 2 mm thick, weighed 45 mg, and had a hardness of 1-3.5 Kp.

The outer annular body was made by mixing for 5 minutes 48.5 parts Nu-Tab™, 45 parts of Microcellac 100™, 0.5 parts of sodium saccharin, and 5 parts of crospovidone. Thereafter, one part magnesium stearate was added and mixed for half of a minute to obtain a final mixture. The final mixture was compressed into tablets using a Manesty f3 tablet press fitted with a set of tooling such as that described in U.S. publication No. 2003/206,954 and PCT Publication No. WO 03/057136. Each tablet weighed 290 mg. The tablet outer diameter was 9 mm, height about 4.5 mm, and the hardness was 5-9 Kp.

Pharmacokinetic Trial

In a crossover study, twelve volunteer subjects were administered a 4 mg commercial oral preparation of tizanidine (Zanaflex™) and a 2 mg sublingual tablet formulated as described herein. Two groups were randomized and there was a one week washout period between administrations. The volunteers were in the fasted state when the drugs were administered. The sublingual tablets were placed under the tongue for 5 minutes and tablet remnants, if any, were swallowed. The oral formulation was administered with a glass of water. Blood samples were taken at 0, 0.5, 1.0, 1.5, 2.0, 2.5, 3.0, 4.0, 5.0, 6.0, and 7.0 hours after administration. The plasma was separated from the whole blood and the tizanidine concentration was determined by a validated HPLC assay. The samples were blinded from the analysts. All twelve volunteers participated in the sublingual study while one volunteer did not participate in the oral delivery study.

Results

Table 1 summarizes the plasma tizanidine levels for twelve test subjects who were administered 2 mg tizanidine in a sublingual formulation.

Table 2 summarizes the data for 11 of the twelve test subjects (test subject 6 did not participate in the oral delivery study) who were administered 4 mg tizanidine in a standard commercial oral formulation.

Table 3 presents the calculated pharmacokinetic parameters for both groups in Table 1 and Table 2.

TABLE 1
Plasma Tizanidine Levels (pg/g) after Sublingual Delivery of Tizanidine (2 mg)

| | Test Subject No. | | | | | | | | | | | |
|---|---|---|---|---|---|---|---|---|---|---|---|---|
| Time | 1 | 2 | 3 | 4 | 5 | 6 | 7 | 8 | 9 | 10 | 11 | 12 |
| 0 | <98.40 | NRV | <98.40 | <98.40 | <98.40 | <98.40 | <98.40 | <98.40 | <98.40 | <98.40 | <98.40 | <98.40 |
| 0.5 | 775.02 | 532.00 | 1553.79 | 145.17 | 1896.26 | 776.45 | 443.45 | 1431.47 | 313.12 | 1961.88 | 471.87 | 245.53 |
| 1 | 1278.80 | 988.08 | 1459.29 | 481.79 | 2019.72 | 1197.3 | 1034.11 | 1973.34 | 567.10 | 1431.73 | 973.92 | 952.86 |
| 1.5 | 972.00 | 924.2 | 998.88 | 513.86 | 1691.20 | 824.94 | 1845.35 | 1963.51 | 741.25 | 1079.73 | 1055.64 | 1021.82 |
| 2 | 788.93 | NRV | 990.63 | 766.84 | 1618.61 | 548.21 | 1832.81 | 1471.67 | 1880.60 | 995.41 | 660.18 | 675.70 |
| 2.5 | 560.71 | 643.96 | 838.46 | 639.48 | 935.25 | 390.09 | 1721.56 | 968.31 | 1025.96 | 609.41 | 414.75 | 506.19 |
| 3 | 341.03 | 467.46 | 758.47 | 471.44 | 874.76 | 275.99 | 1447.37 | 650.90 | 585.00 | 519.01 | 301.19 | 294.73 |
| 4 | 245.64 | 375.05 | 472.62 | 308.53 | 479.04 | 170.75 | 866.4 | 403.68 | 357.36 | 264.30 | 152.11 | 162.55 |
| 5 | 110.77 | 244.92 | 282.94 | 323.53 | 304.18 | 137.31 | 749.04 | 243.47 | 270.23 | 171.07 | <98.40 | 116.40 |
| 6 | <98.40 | NRV | NRV | 145.36 | 253.06 | <98.40 | 489.89 | 184.65 | 183.24 | 118.64 | <98.40 | <98.40 |
| 7 | <98.40 | 101.95 | 117.57 | <98.40 | 183.79 | <98.40 | 389.01 | 100.10 | <98.40 | <98.40 | <98.40 | <98.40 |

NRV = No value reported.

TABLE 2
Plasma Tizanidine Levels (pg/g) after Gastric Delivery of Tizanidine
(4 mg) in Commercial Immediate Release Formulation

| | Test Subject No. | | | | | | | | | | |
|---|---|---|---|---|---|---|---|---|---|---|---|
| Time | 1 | 2 | 3 | 4 | 5 | 7 | 8 | 9 | 10 | 11 | 12 |
| 0 | <98.40 | <98.40 | <98.40 | <98.40 | <98.40 | <98.40 | <98.40 | <98.40 | <98.40 | <98.40 | <98.40 |
| 0.5 | 111.53 | NRV | 2750.73 | 143.68 | 375.93 | 1581.27 | 1442.94 | 417.87 | 1680.10 | 2336.94 | 812.94 |
| 1 | 1263.88 | 1149.86 | 2164.91 | 525.09 | 5715.05 | 2248.04 | 3417.33 | 2513.57 | 1515.56 | 1395.25 | 1504.56 |
| 1.5 | 1263.59 | 1673.44 | 1608.80 | 873.90 | 3990.80 | 2991.92 | 2971.57 | 1306.68 | 1181.31 | 1058.19 | 1344.10 |
| 2 | 817.28 | 1934.76 | 1282.80 | 1086.72 | 3111.55 | 2471.10 | 2829.05 | 890.87 | 782.03 | 847.13 | 1150.98 |
| 2.5 | 711.36 | 1406.92 | 970.04 | 1138.61 | 2224.19 | 3448.21 | 3164.19 | 672.94 | 517.33 | 490.23 | 823.30 |
| 3 | 434.83 | 965.01 | 818.19 | 447.45 | 1787.40 | 2802.43 | 2016.60 | 419.12 | 360.95 | 454.67 | 523.06 |
| 4 | 198.21 | 577.89 | 477.16 | 305.11 | 1348.44 | 1933.08 | 1121.49 | 197.08 | 218.69 | 316.96 | 336.86 |
| 5 | 170.07 | NRV | 301.57 | 272.98 | 962.62 | 1209.45 | 964.26 | 180.33 | 161.26 | 230.63 | 159.62 |
| 6 | <98.40 | NRV | 292.12 | <98.40 | 438.65 | 704.52 | 580.47 | <98.40 | <98.40 | 147.53 | 99.22 |
| 7 | <98.40 | <98.40 | 150.48 | 108.18 | 464.21 | 363.02 | 212.05 | <98.40 | <98.40 | <98.40 | <98.40 |

NRV = No value reported.

TABLE 3
Summary of Pharmacokinetic Data for 2 mg Sublingual (Test) v. 4 mg Oral (Ref.)

| | AUC (h*pg/g) | | AUC_{inf} (h*pg/g) | | t_{1/2} (h) | | Tₘₐₓ (h) | | Cₘₐₓ (pg/g) | |
|---|---|---|---|---|---|---|---|---|---|---|
| Subject # | Test | Reference | Test | Reference | Test | Reference | Test | Reference | Test | Reference |
| 1 | 2799.9 | 2778.2 | 2799.9 | 2778.2 | 1.1 | 1.2 | 1.0 | 1.0 | 1278.8 | 1263.9 |
| 2 | 3110.7 | — | 3393.0 | — | 1.9 | — | 1.0 | 2.0 | 988.1 | 1934.8 |
| 3 | 4503.7 | 6148.4 | 4783.8 | 6536.5 | 1.7 | 1.8 | 0.5 | 0.5 | 1553.8 | 2750.7 |
| 4 | 2404.4 | 2851.8 | 2404.4 | 3289.6 | 1.9 | 2.8 | 2.0 | 2.5 | 766.8 | 1138.6 |
| 5 | 5882.8 | 12031.1 | 6366.6 | 13246.5 | 1.8 | 1.8 | 1.0 | 1.0 | 2019.7 | 5715.1 |
| 6 | 2383.6 | — | 2383.6 | — | 1.5 | — | 1.0 | — | 1197.4 | — |
| 7 | 6824.0 | 12500.7 | 8040.4 | 13153.8 | 2.2 | 1.2 | 1.5 | 2.5 | 1845.4 | 3448.2 |
| 8 | 5724.2 | 11197.2 | 5483.9 | 11607.9 | 1.5 | 1.3 | 1.0 | 1.0 | 1973.3 | 3417.3 |
| 9 | 3513.6 | 3592.6 | 3513.6 | 3592.6 | 1.8 | 1.1 | 2.0 | 1.0 | 1880.6 | 2513.3 |
| 10 | 3982.3 | 3488.9 | 3982.3 | 3488.9 | 1.3 | 1.3 | 0.5 | 0.5 | 1961.9 | 1680.1 |
| 11 | 2166.2 | 4100.0 | 2166.2 | 4100.0 | 1.0 | 1.8 | 1.5 | 0.5 | 1055.6 | 2336.9 |
| 12 | 2201.0 | 3805.9 | 2201.0 | 3805.9 | 1.1 | 1.2 | 1.5 | 1.0 | 1021.8 | 1504.6 |
| AVG | 3753.9 | 6249.5 | 3959.9 | 6560.0 | 1.6 | 1.6 | 1.3 | 1.2 | 1461.9 | 2518.5 |
| geomn | 3481.9 | 5254.5 | 3608.7 | 5462.0 | 1.5 | 1.5 | 1.1 | 1.0 | 1391.6 | 2254.8 |
| s.e. | 451.4 | 1162.1 | 540.2 | 1256.6 | 0.1 | 0.1 | 0.1 | 0.2 | 132.7 | 382.2 |
| s.d. | 1564 | 4026 | 1871 | 4353 | 0.4 | 0.5 | 0.5 | 0.8 | 460 | 1324 |
| r.s.d. | 0.4166 | 0.6442 | 0.4725 | 0.6636 | — | — | — | — | — | — |
| Δ % r.s.d. | −35.3 | — | −28.8 | — | — | — | — | — | — | — |

The average total amount absorbed (the area under the plasma concentration vs. time curve extrapolated to infinity, AUC_{inf}) was 6560 for the 4 mg oral tablet while the result was 3960 for the 2 mg sublingual tablet. Normalizing the dose data gave 1640/mg for the oral delivery and 1980/mg for the sublingual delivery, reflecting a 20% increase in bioavailability using the sublingual delivery system. The average Cₘₐₓ for the 2 mg sublingual delivery was 1462 (731/mg), in contrast the Cₘₐₓ for the 4 mg oral dose was 2519 (630/mg). Thus, the sublingual delivery system gave a Cₘₐₓ that was about 16% higher. The standard deviation of the AUC for the oral formulation was 4353 (relative standard deviation of 66%) while the standard deviation of the AUC for the 2 mg sublingual formulation was 1871 (relative standard deviation of 47%) reflecting a decrease in variation of 28.8%. Therefore, the data for the study demonstrated that sublingual and buccal delivery gave less variability in results and improved bioavailability as compared to conventional oral delivery where the drug is absorbed in the intestine. The half life of the tizanidine was found to be 1.5 to 1.7 hours.

### Example 2

A randomized 4-way four-period crossover ascending dose comparative bioavailability study was conducted using three doses of sublingual tizanidine HCl and one oral Zanaflex® (Tizanidine HCl, 4 mg) tablet in healthy male volunteers. The study was a randomized open label study with a four period comparative crossover study. Blood samples (5 ml) were taken to determine tizanidine plasma concentrations. The blood samples were taken at “0” hour (pre-dosing), 10 min, 20 min, 40 min, 1.0. 1.5, 2.0, 2.5, 3.0, 3.5, 4.0, 5.0, 6.0, 7.0, 9.0 and 12.0 hours post-initial dosing for a total of 16 blood samples per study period.

The study contained twelve (12) male subjects aged 18-55 years. One subject, Subject 10 (I-R), did not participate in study period 4 (Test 2, 4 mg sublingual tablet) due to adverse events (abdominal pain and diarrhoea). In summary, eleven (11) subjects completed the study as planned. However the available pharmacokinetic data from all twelve (12) subjects was included in the statistical analysis, as required by the study protocol.

The study administered the single dosages in four different forms. Administration 1 (A) comprised sublingually administering tizanidine HCL (2 mg) sublingual tablet (“Test 1”). Administration 2 (B) comprised sublingually administering tizanidine HCL (4 mg) sublingual tablet (“Test 2”). Administration 3 (C) comprised sublingually administering tizanidine HCL (8 mg) sublingual tablet (“Test 3”). Administration 4 (D) comprised orally administering commercially available Zanaflex® oral tablet (4 mg; Athena Neurosciences) (“Reference”).

Safety Results:

The data demonstrates that there was a dose related reduction in mean supine systolic blood pressure following administration of the three Test formulations. Also, there was a similar reduction in mean supine systolic blood pressure between Test 2 (4 mg sublingual tablet) and the Reference (4 mg oral tablet). However no clinical consequences were noted with regard to any increase in adverse event reporting.

A total of thirteen (13) adverse events were reported by eight (8) subjects during the study. One (1) adverse event (headache) was reported by one subject before dosing. One (1) adverse event (headache) was reported by one subject following treatment with Test 1 (sublingual, 2 mg). The headache was classified as mild in severity, considered not related to the study medication and resolved without treatment. One (1) adverse event (chest pain) was reported by one subject following treatment Test 2 (sublingual, 4 mg). This was classified as mild in severity, considered unlikely to be related to the study medication and resolved without treatment. Six (6) adverse events were reported by three (3) subjects following treatment Test 3 (sublingual, 8 mg). However, four (4) of these were reported by a single subject (diarrhoea, asthenia, rhinitis, and pharyngitis). All four were classified as mild in severity, considered not related to the study medication and resolved without treatment. The remaining two adverse events were also classified as mild in severity. One (back pain) was considered not related to the study medication and resolved following treatment with diclofenac ointment. The other (asthenia) was considered possibly related to the study medication and resolved without treatment.

Four (4) adverse events were reported by two (2) subjects following treatment with the Reference (oral, 4 mg). Two (both headache) were reported by one subject. Both of these were classified as mild in severity and not related to the study medication. One event was resolved without treatment while the other was resolved following treatment with paracetamol. The other two (2) adverse events (abdominal pain and diarrhoea) were reported by one subject. Both of these were classified as moderate in severity and unlikely to be related to the study medication, and resulted in the subject being withdrawn from the study.

Pharmacokinetic Results

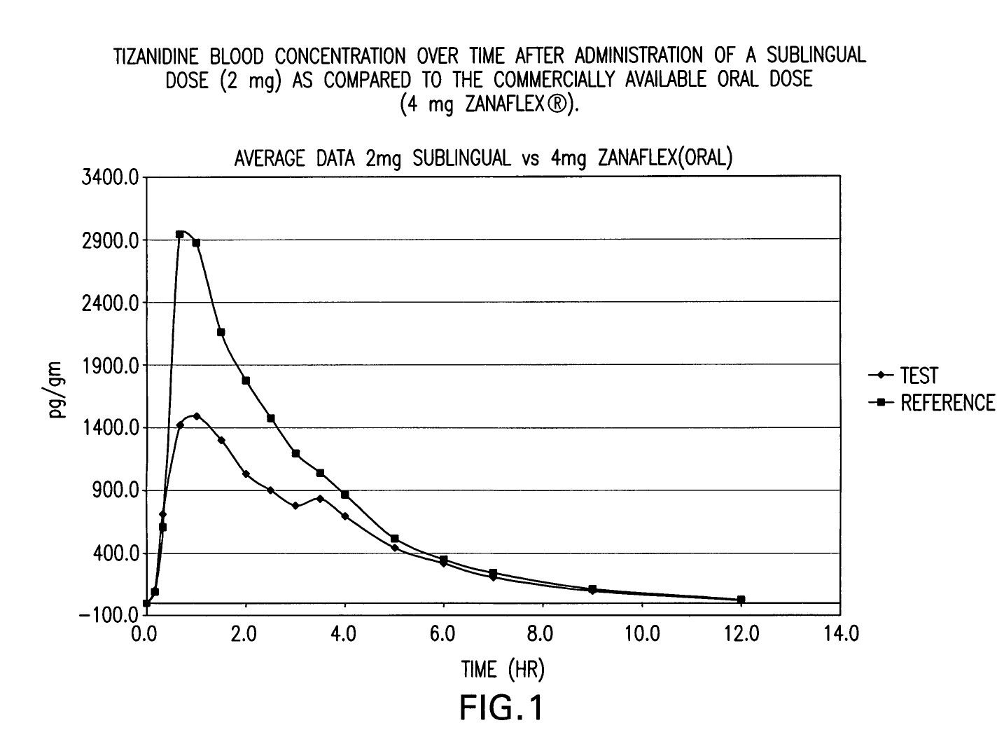

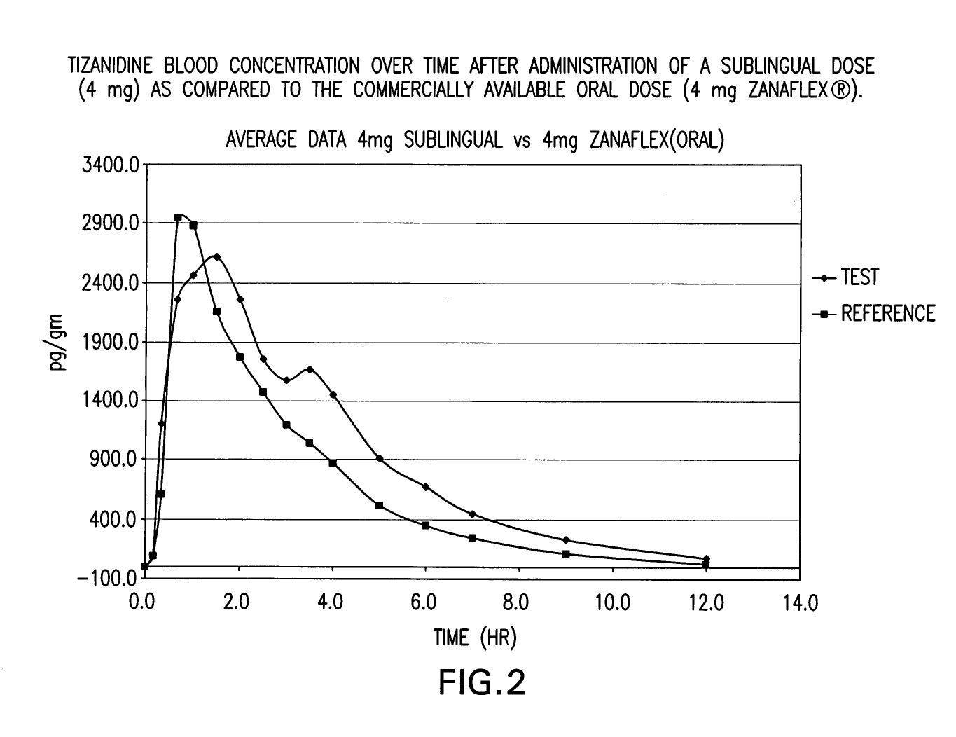

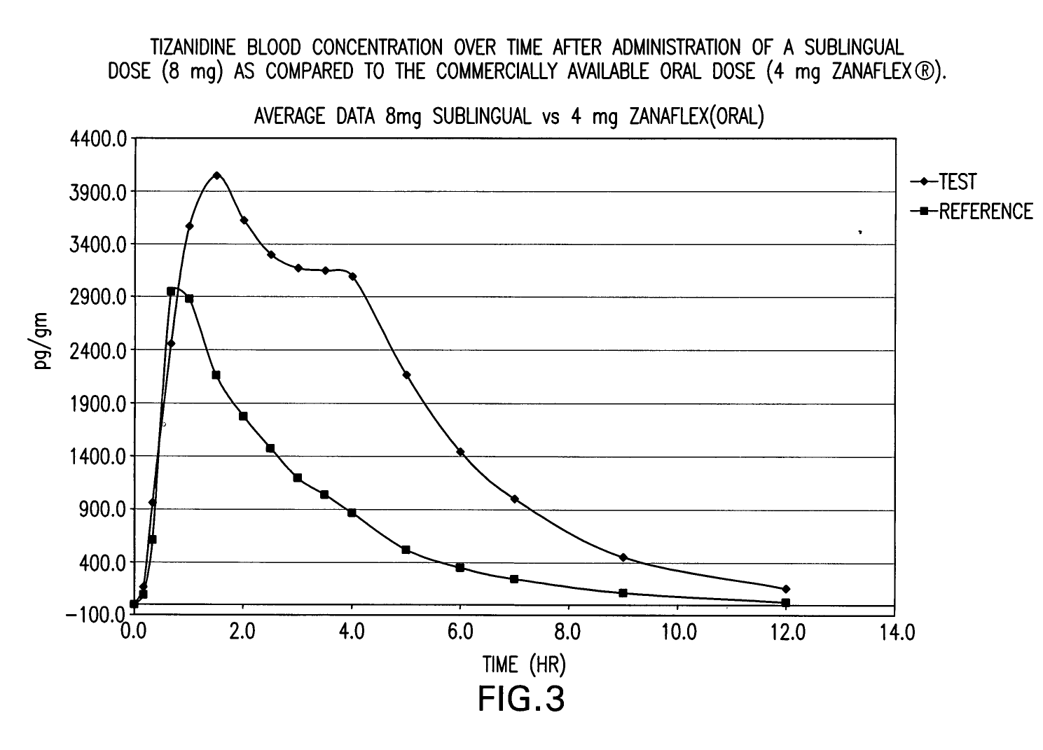

Tables 4, 5, and 6 summarize the pharmacokinetic results of the 2 mg, 4 mg, and 8 mg, dosage forms as compared to the reference sample. FIGS. 1, 2, and 3 graphically represent the data of tables 4-6. The graph is data averaged per time point so values are not identical to the averages in the tables which are averaged over the individual volunteers.

TABLE 4
Results of Test Sublingual Tizanidine (2 mg) and Reference Oral Tizanidine (4 mg)

| | AUC (h*pg/g) | | AUC_{inf} (h*pg/g) | | t_{1/2} (h) | | CL | | Vd | | Tₘₐₓ (h) | | Cₘₐₓ (pg/g) | | Cₘₐₓ | AUC |
|---|---|---|---|---|---|---|---|---|---|---|---|---|---|---|---|---|
| No. | Test | Ref. | Test | Ref. | Test | Ref | Test | Ref. | Test | Ref. | Test | Ref. | Test | Ref. | ratio | ratio |
| 1 | 9306.3 | 14087.1 | 9462.3 | 14444.9 | 1.8 | 2.3 | 214.9 | 283.9 | 570.5 | 956.5 | 1.50 | 0.67 | 2929.2 | 3565.8 | 0.82 | 0.66 |
| 2 | 4361.7 | 9151.2 | 4361.7 | 9151.2 | 1.8 | 1.7 | 458.5 | 437.1 | 1173.5 | 1071.1 | 1.00 | 0.67 | 1463.1 | 3799.2 | 0.39 | 0.48 |
| 3 | 2180.7 | 3201.0 | 2180.7 | 3201.0 | 1.8 | 1.2 | 917.1 | 1249.6 | 2386.7 | 2172.7 | 1.00 | 0.67 | 838.0 | 1987.3 | 0.42 | 0.68 |
| 4 | 6093.6 | 6744.4 | 6093.6 | 6744.4 | 1.6 | 1.6 | 328.2 | 593.1 | 753.4 | 1381.0 | 0.67 | 0.67 | 2543.0 | 3868.0 | 0.66 | 0.90 |
| 5 | 4574.0 | 3359.5 | 4574.0 | 3359.5 | 1.3 | 1.1 | 437.3 | 1190.6 | 814.9 | 1859.3 | 1.00 | 1.00 | 1640.4 | 1557.3 | 1.05 | 1.36 |
| 6 | 12405.6 | 15146.2 | 12761.1 | 15404.3 | 2.1 | 1.9 | 161.2 | 264.1 | 486.5 | 720.5 | 2.00 | 1.00 | 3542.4 | 5219.6 | 0.68 | 0.83 |
| 7 | 6582.8 | 7021.6 | 6582.8 | 7021.6 | 2.0 | 1.9 | 303.8 | 569.7 | 891.7 | 1557.6 | 0.33 | 0.67 | 3141.2 | 3195.8 | 0.98 | 0.94 |
| 8 | 2181.2 | 8303.2 | 2181.2 | 8303.2 | 1.7 | 1.9 | 916.9 | 481.7 | 2285.0 | 1307.8 | 1.00 | 0.67 | 746.6 | 3919.3 | 0.19 | 0.26 |
| 9 | 4560.5 | 4708.3 | 4560.5 | 4708.3 | 1.4 | 1.4 | 438.5 | 849.6 | 891.2 | 1772.8 | 1.50 | 1.00 | 1392.8 | 2437.3 | 0.57 | 0.97 |
| 10 | 6525.8 | 7872.0 | 6525.8 | 7872.0 | 1.6 | 1.7 | 306.5 | 508.1 | 727.4 | 1254.8 | 0.67 | 1.00 | 2817.7 | 3333.8 | 0.85 | 0.83 |
| 11 | 4490.3 | 5120.5 | 4490.3 | 5120.5 | 1.6 | 2.0 | 445.4 | 781.2 | 1060.3 | 2233.3 | 1.50 | 1.00 | 1771.9 | 2349.6 | 0.75 | 0.88 |
| 12 | 10082.1 | 12286.1 | 10082.1 | 12286.1 | 1.5 | 1.7 | 198.4 | 325.6 | 426.0 | 791.6 | 2.00 | 1.00 | 2979.6 | 4511.0 | 0.66 | 0.82 |
| AVG | 6112.0 | 8083.4 | 6154.7 | 8134.8 | 1.7 | 1.7 | 427.2 | 627.9 | 1038.9 | 1423.2 | 1.0 | 0.8 | 2150.5 | 3312.0 | 0.669 | 0.800 |
| geomn | 5383.1 | 7195.1 | 5403.3 | 7220.4 | 1.7 | 1.7 | 371.5 | 555.9 | 900.6 | 1338.2 | 1.1 | 0.8 | 1924.4 | 3138.4 | 0.613 | 0.748 |
| stddev | 3124 | 3978 | 3204 | 4070 | 0.2 | 0.3 | 250.3 | 329.5 | 643.9 | 506.9 | 0.5 | 0.2 | 951 | 1070 | 0.250 | 0.271 |
| std-err | 901.8 | | 925.0 | | 0.1 | | 72.3 | | 185.9 | | 0.2 | | 274.6 | | | |

TABLE 5
Results of Test Sublingual Tizanidine (4 mg) and Reference Oral Tizanidine (4 mg)

| | AUC (h*pg/g) | | AUC_{inf} (h*pg/g) | | t_{1/2} (h) | | CL | | Vd | | Tₘₐₓ (h) | | Cₘₐₓ (pg/g) | | Cₘₐₓ | AUC |
|---|---|---|---|---|---|---|---|---|---|---|---|---|---|---|---|---|
| No. | Test | Ref. | Test | Ref. | Test | Ref | Test | Ref. | Test | Ref. | Test | Ref. | Test | Ref. | Test | Ref |
| 1 | 17984.2 | 14087.1 | 18392.1 | 14444.9 | 2.0 | 2.3 | 222.4 | 283.9 | 635.4 | 956.5 | 1.50 | 0.67 | 5051.7 | 3565.8 | 1.42 | 1.27 |
| 2 | 11712.6 | 9151.2 | 11878.4 | 9151.2 | 1.9 | 1.7 | 341.5 | 437.1 | 920.6 | 1071.1 | 1.50 | 0.67 | 3911.1 | 3799.2 | 1.03 | 1.30 |
| 3 | 5245.2 | 3201.0 | 5245.2 | 3201.0 | 1.3 | 1.2 | 762.6 | 1249.6 | 1483.9 | 2172.7 | 1.50 | 0.67 | 1493.8 | 1987.3 | 0.75 | 1.64 |
| 4 | 14833.5 | 6744.4 | 14958.5 | 6744.4 | 1.7 | 1.6 | 269.7 | 593.1 | 662.9 | 1381.0 | 0.67 | 0.67 | 5050.5 | 3868.0 | 1.31 | 2.22 |
| 5 | 10622.5 | 3359.5 | 10622.5 | 3359.5 | 1.4 | 1.1 | 376.6 | 1190.6 | 776.1 | 1859.3 | 0.67 | 1.00 | 3908.0 | 1557.3 | 2.51 | 3.16 |
| 6 | 20051.2 | 15146.2 | 20553.5 | 15404.3 | 2.1 | 1.9 | 199.5 | 264.1 | 598.1 | 720.5 | 1.00 | 1.00 | 4924.3 | 5219.6 | 0.94 | 1.33 |
| 7 | 13447.5 | 7021.6 | 13999.1 | 7021.6 | 2.3 | 1.9 | 297.5 | 569.7 | 984.5 | 1557.6 | 1.00 | 0.67 | 2859.2 | 3195.8 | 0.89 | 1.99 |
| 8 | 3995.6 | 8303.2 | 3995.6 | 8303.2 | 1.7 | 1.9 | 1001.1 | 481.7 | 2419.7 | 1307.8 | 1.50 | 0.67 | 1334.0 | 3919.3 | 0.34 | 0.48 |
| 9 | 10561.1 | 4708.3 | 10561.1 | 4708.3 | 1.7 | 1.4 | 378.7 | 849.6 | 920.1 | 1772.8 | 1.50 | 1.00 | 2920.8 | 2437.3 | 1.20 | 2.24 |
| 10 | | 7872.0 | | 7872.0 | | 1.7 | | 508.1 | | 1254.8 | | 1.00 | | 3333.8 | | |
| 11 | 7721.5 | 5120.5 | 7721.5 | 5120.5 | 1.6 | 2.0 | 518.0 | 781.2 | 1209.5 | 2233.3 | 2.50 | 1.00 | 2041.6 | 2349.6 | 0.87 | 1.51 |
| 12 | 17953.2 | 12286.1 | 17953.2 | 12286.1 | 1.4 | 1.7 | 222.8 | 325.6 | 454.0 | 791.6 | 2.50 | 1.00 | 4727.2 | 4511.0 | 1.05 | 1.46 |
| AVG | 12193.5 | 8083.4 | 12352.8 | 8134.8 | 1.7 | 1.7 | 417.3 | 627.9 | 1005.9 | 1423.2 | 1.1 | 0.8 | 3474.7 | 3312.0 | 1.119 | 1.692 |
| geomn | 10961.8 | 7195.1 | 11071.8 | 7220.4 | 1.7 | 1.7 | 364.9 | 555.9 | 902.0 | 1338.2 | 1.3 | 0.8 | 3155.1 | 3138.4 | 1.001 | 1.546 |
| stddev | 5271 | 3978 | 5411 | 4070 | 0.3 | 0.3 | 252.7 | 329.5 | 553.4 | 506.9 | 0.6 | 0.2 | 1424 | 1070 | 0.554 | 0.696 |
| std-err | 1589.3 | 1148.3 | 1631.6 | 1174.8 | 0.1 | 0.1 | 76.2 | 95.1 | 166.9 | 146.3 | 0.2 | 0.1 | 429.2 | 308.9 | | |

TABLE 6
Results of Test Sublingual Tizanidine (8 mg) and Reference Oral Tizanidine (4 mg)

| | AUC (h*pg/g) | | AUC_{inf} (h*pg/g) | | t_{1/2} (h) | | CL | | Vd | | Tₘₐₓ (h) | | Cₘₐₓ (pg/g) | | Cₘₐₓ | AUC |
|---|---|---|---|---|---|---|---|---|---|---|---|---|---|---|---|---|
| No. | Test | Ref. | Test | Ref. | Test | Ref | Test | Ref. | Test | Ref. | Test | Ref. | Test | Ref. | Test | Ref |
| 1 | 26490.1 | 14087.1 | 27328.3 | 14444.9 | 2.2 | 2.3 | 302.0 | 283.9 | 943.4 | 956.5 | 1.50 | 0.67 | 7020.4 | 3565.8 | 1.97 | 1.89 |
| 2 | 18780.3 | 9151.2 | 19114.8 | 9151.2 | 1.8 | 1.7 | 426.0 | 437.1 | 1123.8 | 1071.1 | 2.00 | 0.67 | 4388.6 | 3799.2 | 1.16 | 2.09 |
| 3 | 11178.6 | 3201.0 | 11178.6 | 3201.0 | 1.5 | 1.2 | 715.7 | 1249.6 | 1575.1 | 2172.7 | 1.00 | 0.67 | 3021.0 | 1987.3 | 1.52 | 3.49 |
| 4 | 23426.9 | 6744.4 | 23775.9 | 6744.4 | 1.8 | 1.6 | 341.5 | 593.1 | 878.5 | 1381.0 | 2.00 | 0.67 | 7217.7 | 3868.0 | 1.87 | 3.53 |
| 5 | 15107.3 | 3359.5 | 15212.5 | 3359.5 | 1.3 | 1.1 | 529.5 | 1190.6 | 990.5 | 1859.3 | 2.00 | 1.00 | 3619.9 | 1557.3 | 2.32 | 4.53 |
| 6 | 34828.8 | 15146.2 | 35916.7 | 15404.3 | 1.8 | 1.9 | 229.7 | 264.1 | 604.6 | 720.5 | 3.50 | 1.00 | 5872.7 | 5219.6 | 1.13 | 2.33 |
| 7 | 22125.0 | 7021.6 | 22598.6 | 7021.6 | 1.9 | 1.9 | 361.6 | 569.7 | 966.9 | 1557.6 | 1.50 | 0.67 | 4160.4 | 3195.8 | 1.30 | 3.22 |
| 8 | 8481.3 | 8303.2 | 8606.0 | 8303.2 | 1.7 | 1.9 | 943.2 | 481.7 | 2321.2 | 1307.8 | 0.67 | 0.67 | 2580.8 | 3919.3 | 0.66 | 1.04 |
| 9 | 21695.1 | 4708.3 | 21858.0 | 4708.3 | 1.4 | 1.4 | 368.7 | 849.6 | 736.3 | 1772.8 | 1.50 | 1.00 | 6359.3 | 2437.3 | 2.61 | 4.64 |
| 10 | 19690.4 | 7872.0 | 19968.8 | 7872.0 | 1.8 | 1.7 | 406.3 | 508.1 | 1063.4 | 1254.8 | 0.67 | 1.00 | 7150.4 | 3333.8 | 2.14 | 2.54 |
| 11 | 17529.0 | 5120.5 | 17729.1 | 5120.5 | 1.6 | 2.0 | 456.4 | 781.2 | 1038.5 | 2233.3 | 1.50 | 1.00 | 5374.5 | 2349.6 | 2.29 | 3.46 |
| 12 | 32500.8 | 12286.1 | 32778.5 | 12286.1 | 1.6 | 1.7 | 246.1 | 325.6 | 569.7 | 791.6 | 1.50 | 1.00 | 9791.3 | 4511.0 | 2.17 | 2.67 |
| AVG | 20986.1 | 8083.4 | 21338.8 | 8134.8 | 1.7 | 1.7 | 443.9 | 627.9 | 1067.7 | 1423.2 | 1.2 | 0.8 | 5546.4 | 3312.0 | 1.761 | 2.952 |
| geom | 19557.0 | 7195.1 | 19850.0 | 7220.4 | 1.7 | 1.7 | 409.1 | 555.9 | 992.5 | 1338.2 | 1.5 | 0.8 | 5172.7 | 3138.4 | 1.648 | 2.749 |
| stddv | 7792 | 3978 | 8033 | 4070 | 0.2 | 0.3 | 204.6 | 329.5 | 474.1 | 506.9 | 0.8 | 0.2 | 2100 | 1070 | 0.598 | 1.065 |
| std-er | 2349.4 | 1148.3 | 2422.0 | 1174.8 | 0.1 | 0.1 | 61.7 | 95.1 | 143.0 | 146.3 | 0.2 | 0.1 | 633.1 | 308.9 | | |

The sublingual delivery of tizanidine resulted in an improved bioavailability as expressed by AUCₜ or AUC_{I} without a similar rise in the Cₘₐₓ. The expectation is one of a more efficacious product without increased side effects. When comparing the 2 mg sublingual delivery to the oral 4 mg tizanidine, the AUC for the sublingual delivery was about 75% of that of the oral delivery even though the tizanidine dose was only 50% of the oral dosage form. The Cₘₐₓ for the same dosage comparison was about 65% on the average of that of the oral delivery.

A comparison of the results of Test 2 (4 mg sublingual delivery) to the Reference (4 mg oral delivery of tizanidine) demonstrated improved bioavailability. When averaged as a ratio over the individual volunteers, the AUC improved about 52% in the averaged AUC and there was an about 69% improvement in bioavailability. The Cₘₐₓ was only 5% higher for the averaged data and 12% higher for the average of the ratio of the values for the individual volunteers. The data, averaged over the time points, had a lower Cₘₐₓ for the sublingual delivery compared to oral delivery. Importantly, the time that the average data was higher than the posited effective level of 900 pg/ml was 3.5 hours for the oral delivery and over 5 hours for the sublingual delivery.

A comparison of the results of Test 3 (8 mg sublingual delivery) to the Reference (4 mg oral delivery of tizanidine) demonstrated improved bioavailability and a lesser rise in Cₘₐₓ. The ratio of the average bioavailability as expressed as AUC was 2.62 for a dose ratio of 2.0 while the average of the ratios of AUC over the volunteers was 2.95. The ratio of the average Cₘₐₓ was only 1.67 for a dose ratio of 2.0 while the average of the ratios of the individual volunteers was 1.76. The time above 900 pg/ml was over seven hours. The half life of the tizanidine was found to be 1.5 to 1.7 hours.

### Example 3

### Test of Spasticity in Cerebral Palsy (CP) Patients

Two ambulatory adolescents suffering from CP were treated with 4 mg sublingual tizanidine before bed and one non-ambulatory adolescent was treated with 6 mg sublingual tizanidine before bed. Their spasticity was measured using the “Ashworth scale” and the “Timed Up and Go Test” (for the two ambulatory patients). For two, their daytime sleepiness was measured using the “Epworth sleepiness scale.” The three measured parameters are described below:

Ashworth Scale

In the “Ashworth scale”, a numerical scale of 0 to 4 was used with each value having a particular meaning. A value of 0 indicated no increase in tone. A value of 1 indicated a slight increase in tone giving a catch when the limb is moved in flexion or extension. A value of 2 indicated a more marked increase in tone but the limb was easily flexed. A value of 3 indicated a considerable increase in tone, and passive movement was difficult. A value of 4 indicated a limb rigid in flexion or extension. Each leg was tested for hip adductor, knee extensor and knee flexor, providing a total score for the limb. Thereafter, the score of each leg was added to yield a total score. An improvement of one to two units was considered to be clinically significant.

Timed Up and Go Test

In the “Timed Up and Go Test,” each patient was asked to stand up from a standard chair with a seat height of between 40 and 50 cm, walk a 3 m distance at a normal pace, turn, walk back to the chair, and sit down. The timing was measured in seconds from the word “go” and ended when the patient's back touched the backrest of the chair. An improvement of several seconds was considered to be clinically significant.

Epworth Sleepiness Scale

The sleepiness or fatigue was measured using the “Epworth Sleepiness Scale.” The “Epworth Sleepiness Scale” was performed at the end of each visit and used to determine the level of daytime sleepiness. A score of 10 or more was considered sleepy. A score of 18 or more was considered very sleepy. At each visit patients were given a form to assess the level of sleepiness while performing various activities or tasks since the previous visit. A value of 0 indicated that the patient had never dozed or slept during those activities. A value of 1 indicated that the patient had a slight chance of dozing or sleeping. A value of 2 indicated that the patient had a moderate chance of dozing or sleeping. A value of 3 indicated that the patient had a high chance of dozing or sleeping. Based on this scale, each patient was asked to evaluate their chance of dozing or sleeping during several activities. The activities included sitting and reading; watching TV; sitting inactive in a public space; being a passenger in a motor vehicle for an hour or more; lying down in the afternoon; sitting and talking to someone; sitting quietly after lunch (no alcohol); and stopped for a few minutes in traffic while driving. A total score was compiled from all answers. When evaluated by a doctor at noon the next day all three subjects showed improvements in their motor functions. The results are summarized Table 7.

TABLE 7
Results of Test of Spasticity in Cerebral Palsy Patients

| | Ashworth | | Timed up and go | | Epworth | |
|---|---|---|---|---|---|---|
| Patient | before | after | before | after | before | after |
| MW | 9 | 6 | 27 sec | 20 sec | 0 | 0 |
| EC | 17 | 13 | — | — | 0 | 0 |
| OD | 7 | 2 | 21 sec | 16 sec | — | — |

### Example 4

### Clinical Efficacy and Safety Study of a Sublingual Tizanidine HCl for the Treatment of Spasticity in Patients with Cerebral Palsy

A sublingual formulation was developed in which tizanidine is directly absorbed into the systemic circulation and thus bypasses the extensive first-pass enterohepatic circulation. Phase I pharmacokinetic studies evaluating various formulations of sublingual tizanidine relative to oral tizanidine demonstrated that the sublingual formulation exhibited longer residence of the test drug in the blood, i.e., the presence of a significantly higher AUC_{I}, with Cₘₐₓ comparable to, or only slightly greater than that observed for oral dosing.

The study was designed as an open-label, clinical efficacy and safety study and included a dose-titration period prior to maintenance on sublingual tizanidine. All patients reported to the clinic as per study schedule, where the visits included: screening, once-weekly titration visits, and two maintenance visits. At all clinic visits, patients and their diaries were reviewed by the physician. Physician and patient assessments were then completed, blood samples taken as per schedule and modified Ashworth and Epworth assessments were performed. GMFM-66 was evaluated at the beginning of titration and at study termination.

Each patient received a sublingual dosage form of tizanidine HCl in 2, 4, or 6 mg dosage form. This was followed by titration (up to 5 weeks) from the lowest dose of sublingual tizanidine (2 mg), upwards to clinical efficacy (2-4 mg increments every ±7 days) to a maximum of 12 mg/day. The optimum therapeutic sublingual tizanidine dose established at titration was then maintained by all patients for 1 week, followed by 1 week of weaning the patient off tizanidine and closeout examinations.

Each patient was evaluated to determine their progress by the following primary efficacy parameters. (1) Modified Ashworth scores (pre-treatment, titration and for 2 weeks on maintenance treatment) and (2) GMFM-66 (Gross Motor Function Measure) scores, at commencement (pre-treatment) and completion (final treatment). Each patient acted as their own control. Patients were also monitored by secondary efficacy parameters which included: (1) patient and physician assessment, including Clinical Global Impression (CGI) scale and Patient Global Impression (PGI); (2) Barthel Index (ADL); and Timed Up & Go (when applicable). If necessary, evaluations also considered modified Epworth Sleepiness Scale and Sleep Actigraphy.

Each patient was monitored using primary and secondary safety parameters. Primary safety parameters included: (1) Modified Epworth Sleepiness scale; (2) BP; (3) LFT; and Sleep actigraphy. Second safety parameters included all other adverse events and laboratory test results.

Study Site and Patient Population

The patients in the study were selected from a school which is a specialized education institution which primarily serves brain-damaged students. The student population included children of ages ranging from 6-21 years. The school operated as a daycare facility with a multi-disciplinary staff comprising remedial teachers, physiotherapists, occupational therapists, nurses and a physician. The children were transported to the school every morning either by their parents or by special transport services. A graduate program was available for those students above 18 years of age, whereby the school assisted the student to integrate into a suitable job environment. The school provided continuing support and guidance to the student, including weekly internal programs, to help them achieve and maintain a maximum degree of independence.

Study patients were not on any anti-spasticity medications when evaluated for inclusion into the trial. Most cerebral palsy patients did not receive specific medicinal treatment for spasticity as a consequence of the disease syndrome. Treatment in the school was mainly limited to physiotherapy with occasional surgical intervention. Patients were selected by the following criteria. Patients were either male or female ≧12 years of age with spastic cerebral palsy. Each patient had to score a minimum Modified Ashworth screening score of 3 for one or both lower limbs and were Gross Motor Function Classification System (GMFCS) grades 3, 4, or 5 at study entry.

Patients were excluded if they met any of the following criteria. (1) Patient had a history of allergy to tizanidine or any inactive component; (2) there were significant abnormalities in screening clinical laboratory parameters (hematologic, renal and hepatic) or urine Dipstix; (3) the patient had orthopedic surgery within 6 weeks of screening; (4) the patient had concurrent use of oral tizanidine or other anti-spasticity medications e.g. Baclofen, Neurontin; (5) the patient had co-morbid conditions or other neurological disorders that would confound assessment of clinical parameters (e.g. epilepsy); (6) the patient participated in another clinical trial within 30 days of study start; and/or (7) the patients were non-cooperative or parents/legal guardians were unwilling to sign consent form.

Twelve male and female cerebral palsy patients were enrolled. All were above 12 years of age, and had significant spasticity. Two patients were Gross Motor Function Classification System (GMFCS) 3, which means that they could walk indoors or outdoors on a level surface with an assistive mobility device (crutches) (hereinafter referred to as “mobile patients”). They could also climb stairs holding onto a railing. But most patients were graded between GMFCS 4-5, which ranges from levels of function achieved before age 6 and self-mobility using a power wheelchair for mobility (GMFCS 4) through to severely disabled children (GMFCS 5) who had physical impairments which restricted voluntary control of movement. All areas of motor function were limited and were not fully compensated for through the use of adaptive equipment and assistive technology. GMFCS 5 children had no means of independent mobility and were transported. Some children achieved self-mobility using a power wheelchair with extensive adaptations.

Study Design

This trial was a pilot, open-label, clinical efficacy and safety study which measured daytime spasticity and mobility indices. The study was amended to include nighttime actigraphy, with and without treatment.

Upon study commencement, an immediate improvement was noticed in spasticity measurements. However, somnolence, a common side effect, was sufficiently present in the children. This was significant enough for the Primary Investigator, at his own discretion, to prescribe the daytime medication to be taken at nighttime in the hope of allowing patients to develop tolerance to the medication side effect, before reverting back to morning doses. It was noted that while patients were taking night doses (usually around 8-9 PM), unexpectedly the daytime parameters (e.g. spasticity) continued to show significant improvement (measured between approximately 11 AM-1 PM). A decision was made to continue the trial on a single nighttime dose and to record all the efficacy criteria, as per the protocol during the day, at school. It was decided to amend the trial with actigraphy (sleep monitor) in an effort to explain why significant improvements in efficacy parameters were still noted at least 8 hours after tizanidine blood levels should have been insignificant (and therefore sub therapeutic). In order for a baseline comparison of actigraphy, patients continued the trial for an additional week off treatment but with actigraphy monitoring. Table 8 summarizes the trial paradigm.

TABLE 8
Trial Paradigm

| | |
|---|---|
| 1. | Screening |
| 2. | Titration (daytime) |
| 3. | Move to nocturnal dosing |
| 4. | Titration (nighttime) |
| 5. | Maintenance (nighttime) |
| 6. | Actigraphy on maintenance treatment |
| 7. | Actigraphy off treatment |

At all clinic visits, patients, questionnaires and diaries were reviewed by the physician. Physician and patient assessments and modified Ashworth and Epworth assessments were performed. Blood samples were taken at screening, monthly and then at close out. The GMFM-66 was performed at the beginning of titration and at study termination.

Towards the end of the trial, once maintenance levels of nocturnal tizanidine were reached, patients wore actigraphy monitors for one week while on tizanidine maintenance treatment and then one week once off tizanidine treatment. Monitors were worn from lights out until the following morning and this was noted in the patient diary. The results were downloaded to a computer and analyzed with dedicated algorithms by a sleep specialist.

Results

Once patients were enrolled in the trial there was great enthusiasm demonstrated by all. A feeling of newfound attention permeated the patients and their surroundings, including teachers and ancillary staff at the school.

Parent follow-up was by close telephonic contact conducted by the co-investigator; parents also filled out questionnaires and attended group meetings before and after the trial was conducted. Patient daily compliance was consistently good. Before beginning the trial, patients were given placebo tablets to practice and become familiar with the sublingual route of administration. A special effort was undertaken to address difficulty with the administration techniques as some of the more disabled patients may have swallowed the tablets.

The patients were initiated into the trial on an once daily, morning dose, titration scheme. As the trial evolved, patients were moved to the nocturnal dose regimen.

Mobile Patients' Results

Both mobile patients demonstrated a statistically significant improvement in their GMFM's (primary efficacy parameter) as measured at baseline and then at study closeout. There was tremendous improvement in the Ashworth scale (a measurement of spasticity, where a change of one unit is considered significant). The timed up-and-go mobility test also showed a considerable improvement in both patients. A 4 mg nocturnal dose was the most common dosing in the trial. The results for both patients are summarized in Table 9 and Table 10.

TABLE 9
Patient OD

| Week | GMFM | Ashworth | Walk | Dose |
|---|---|---|---|---|
| 1. | 53.8 | 7 | 21 | Baseline |
| 2. | | | | 2 mg |
| 3. | | 7 | 17 | 2 mg Nocturnal |
| 4. | | 2 | 16 | 2 mg Nocturnal |
| 5. | | 2 | 14 | 4 mg Nocturnal |
| 6. | 54.6 | 2 | 14 | 4 mg Nocturnal |

TABLE 10
Patient MW

| Week | GMFM | Ashworth | Walk | Dose |
|---|---|---|---|---|
| 1. | 46.5 | 9 | 27 | Baseline |
| 2. | | 6 | 20 | 2 mg |
| 3. | | 7 | 17 | 4 mg Nocturnal |
| 4. | | 6 | 19 | 6 mg Nocturnal |
| 5. | 51.6 | 5 | 16 | 6 mg Nocturnal |

Daytime administration with examination taking place immediately after Tₘₐₓ, demonstrated the greatest improvement on the spasticity indices. Significant responses were seen in all patients, but the most significant changes were observed in patients with greater mobility and cognitive function. The clinical response was moderated by the side effect of somnolence and one report of muscle weakness.

Nighttime dosage seemed to be the best. Improvement in spasticity was recorded the following day, in all patients who had previously demonstrated a positive response. The most significant responses were seen in patients with greater mobility and cognitive function. No side effects were noted and no residual somnolence was seen either by parents or school staff.

Spasticity:

Lower limb spasticity, which was measured by our Ashworth test, showed a positive response to treatment. The improvement of spasticity following nocturnal treatment was not as pronounced as daytime treatment, however it was still significant. Upper limbs, which were not included in the formal Ashworth test, usually reflected a greater degree of improvement over the lower limbs, possibly due to the fact that although spasticity was present, patients had a greater range of movement and fewer contractions in these limbs as a result of constant use. This response was especially noted in the limbs of those patients who were physically mobile and they consequently demonstrated the biggest improvement in Ashworth scores and clinical efficacy, which was demonstrated by improved mobility.

Tiredness:

Drug-related somnolence from nocturnal dosing was not evident at clinic visits or in teacher reports. Baseline values of tiredness using the Epworth Sleep Scale demonstrated that some of the patients we arriving at school tired. When sublingual tizanidine was administered at night, we found that not only was there no side effect of somnolence the next day, but Epworth results improved over baseline values.

Mobility:

Patients that were mobile (with crutches) were asked to complete a mobility test. The “timed up and go” examination required the patient to rise from a chair walk 3 meters, turn around, return to the chair and sit down. Participating patients demonstrated a 50% improvement in recorded times over the 5 week period.

Sleep:

Positive changes in sleep efficiency criteria were displayed across the diversity of patients. The results suggest that children with CP sleep significantly better while they receive sublingual Tizanidine. The actigraphic measures indicated that on this medication these children slept significantly longer, had higher sleep efficiency, and reduced number of waking periods and sleep-wake transitions. It also suggested that this improvement lasted for the duration of the sleep period, i.e. through all 4 quarters of the night (taking into account normal sleep patterns), until the next morning.

The Effects of Sublingual Tizanidine on Actigaphic Sleep in Children with Cerebral Palsy

Sleep efficiency was an important parameter. In normal patients a score of <90% required workup and treatment. Sleep duration measure the total time from falling asleep to the final awaking the next morning. Sleep minutes were the amount of time actually asleep, while wake minutes were those minutes where the patient lied awake in bed. The number of wake minutes were an accumulation of the total minutes of numerous wake episodes during the night. Table 12 summarizes the results which demonstrated a statistical significance in a the studied population. See below.

Body Posture

One patient used to routinely wake up numerous times at night and request from her parents to alter her body posture. The reduction in spasticity allowed the patient to sufficiently move her limbs and to independently position herself into a more comfortable posture. This improvement had two consequences: (1) the patient was more self-sufficient and could possibly sleep better due to greater efficiency of her nocturnal movements which may reduce postural discomfort, thereby not requiring an arousal; and (2) improvement of the care-givers' quality of life, as they were able to sleep uninterrupted for longer periods of time.

Call Button

Another patient's dexterity improved sufficiently to enable her to reach out to the call button above her bed and selectively press the button when she required help.

Decreased Body Tension

A female patient had undergone progressive deterioration of her spasticity over the preceding 6 months. This had caused her hands to become rigidly immobilized, palms facing forward by the sides of her head. Her wheelchair controls had to be positioned accordingly next to her hands for ease of use to enable independent mobility. During the course of the trial this patient was able to spontaneously lower her hands to her lap, although her preferred posture remained next to her head. This patient also experienced episodes of urinary incontinence during the trial, which progressively improved. It was postulated that urinary control in this patient was in a large part due to spasticity of the urinary sphincters. Tizanidine caused a natural relaxation of the sphincters and the patient had episodic incontinence until bladder control was reestablished.

Increased Attention During Class

Teachers of two patients reported that the patients were more relaxed, attentive and focused during morning classroom activities.

Pain Control

A patient with severe physical disability, complained much less frequently about pain from his affected limbs.

Sleep Report: The Effects of Sublingual Tizanidine on Actigraphic Sleep in Children with Cerebral Palsy

This study assessed the effects of sublingual tizanidine on sleep in children with Cerebral Palsy. Six children were monitored with actigraphy during a week while they were on medication and an additional week when they were not taking the medication. The actigraphic measures suggested that the children slept significantly better while they were on medication.

Protocol

Nine children who suffer from Cerebral Palsy (CP) were originally included in this study. These children were already on a protocol examining the effects of the medication. The children were asked to attach an actigraph to their wrist (or other locations) when they went to bed and to remove it following their morning rise time. They were monitored for a week while they were taking the medication before bedtime and an additional week after the medication was discontinued. One child was not included in the study because of compliance issues and the data of another child could not be retrieved because of a technical problem. A third child provided only two or three nights of actigraphy and was not included in data analysis although this child's raw data is presented below. Therefore, 6 children completed the study.

The actigraph is a wristwatch-like device that monitors movement (Micro-Mini, Ambulatory Monitoring Inc.). The actigraph was set up to work in Zero Crossing Mode with 1-Min-epoch interval which was compatible with validated scoring algorithms. Sadeh et al., “Activity-based sleep-wake identification: An empirical test of methodological issues,” Sleep, 17(3), 201-207 (1994). The scoring algorithm has been validated only with normal healthy children. For further information on the use of actigraphy in sleep research see: Ancoli-Israel et al., “The role of actigraphy in the study of sleep and circadian rhythms,” Sleep, 26(3), 342-392 (2003); Sadeh et al., “The role of actigraphy in sleep medicine.” Sleep Medicine Reviews, 6(2), 113-124 (2002); Sadeh et al., “The role of actigraphy in the evaluation of sleep disorders,” Sleep, 18(4), 288-302 (1995); and Sadeh et al., (1994).

Data Analysis

Data files were viewed visually and compared to the sleep schedules reported by the parents. Suspected nights with significant discrepancies (more than 30 minutes) or with no complementary parental reports were excluded from data analysis. All decisions about exclusion of nights were based on credibility of the data and were done prior to the final data analysis. Table 11 summarizes the number of nights analyzed.

TABLE 11
Number of Nights Analyzed for Each Child

| Patient | Off Treatment | Treatment |
|---|---|---|
| A | 7 | 3 |
| E | 4 | 4 |
| L | 5 | 3 |
| M | 3 | 7 |
| R | 7 | 6 |
| Z | 2 | 6 |

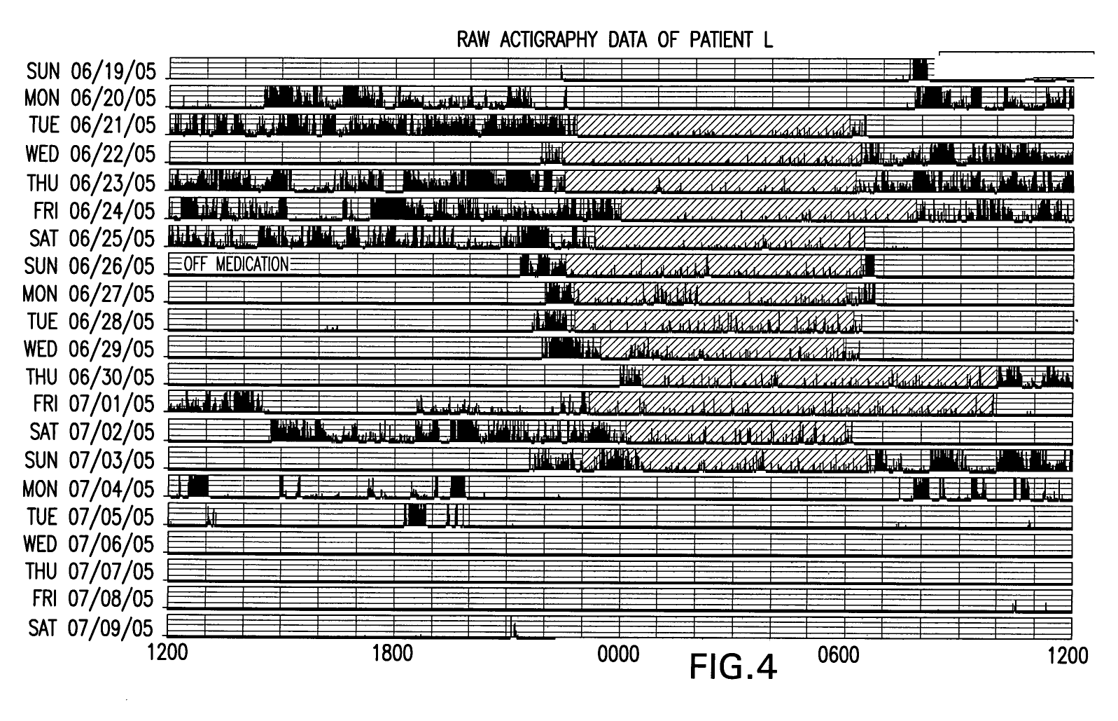

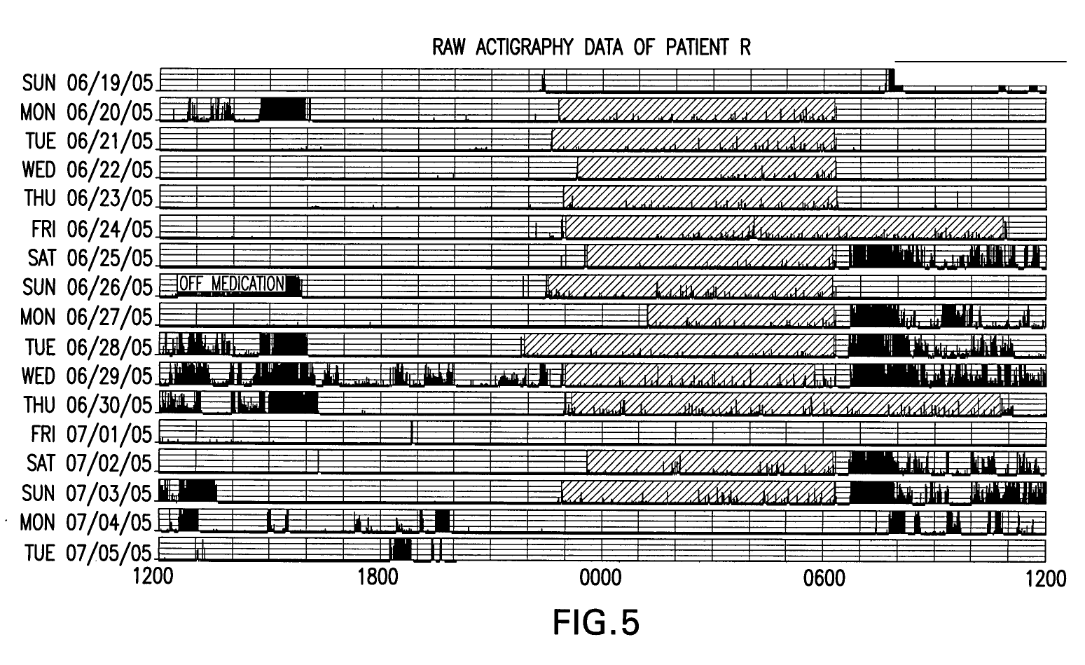

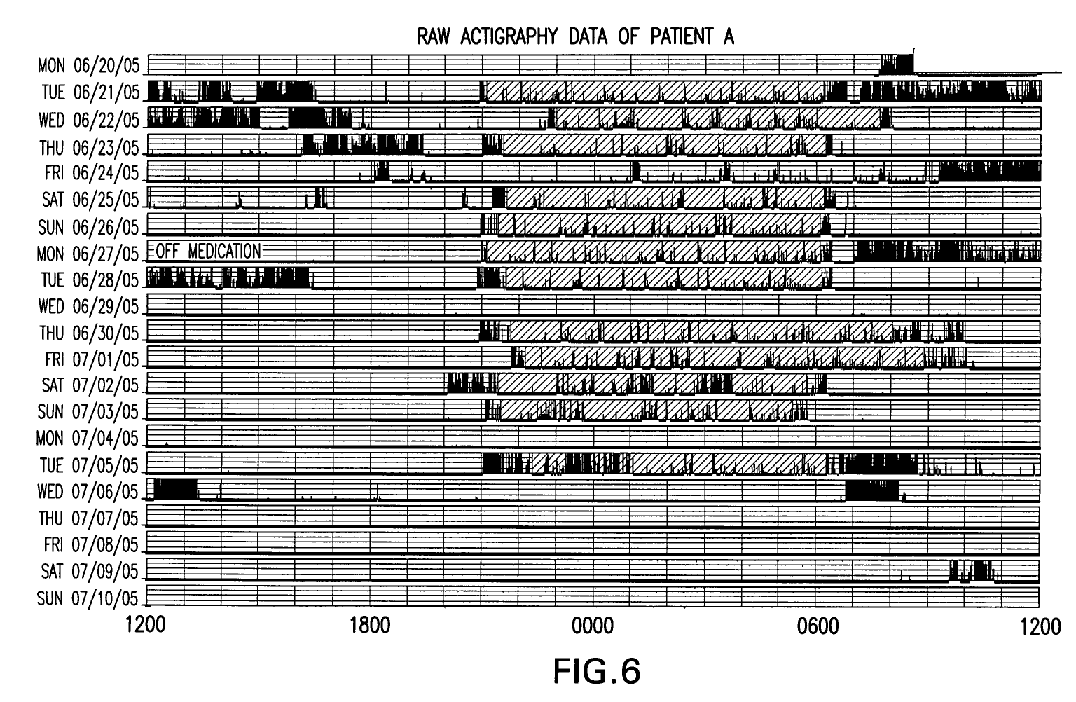

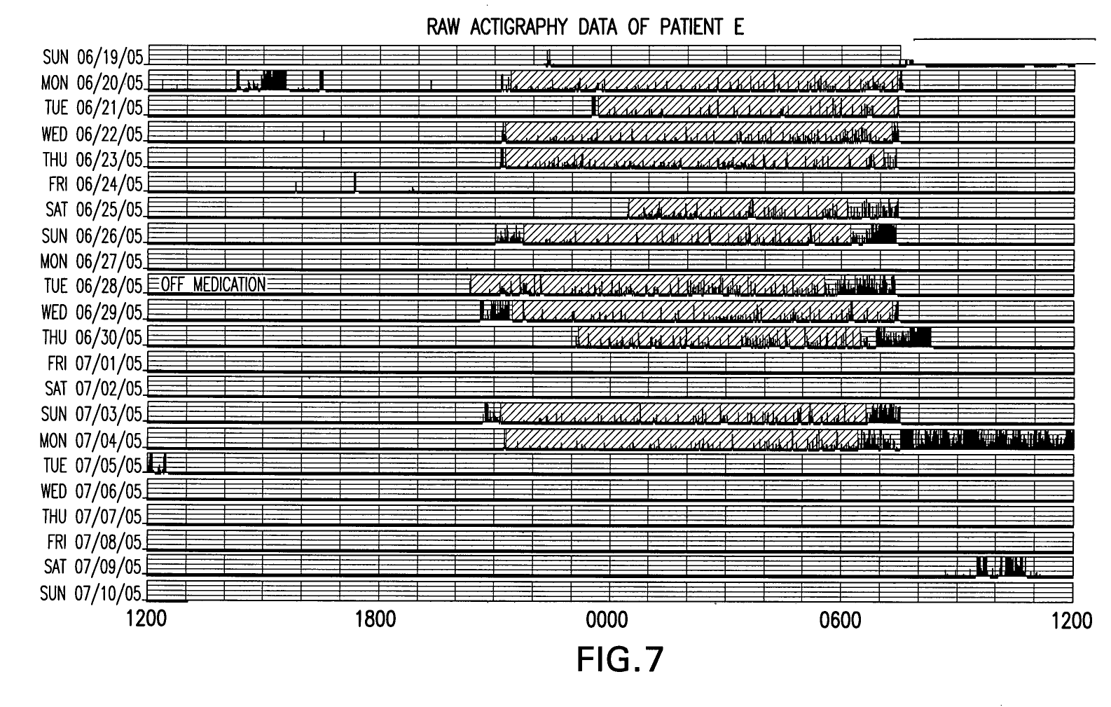

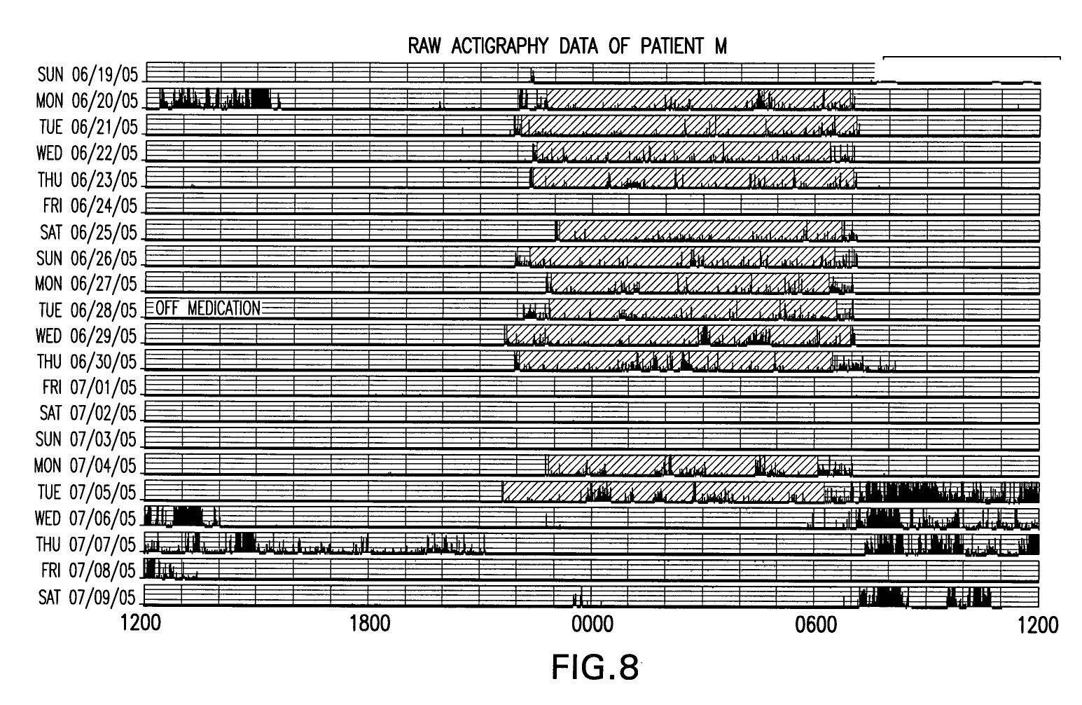

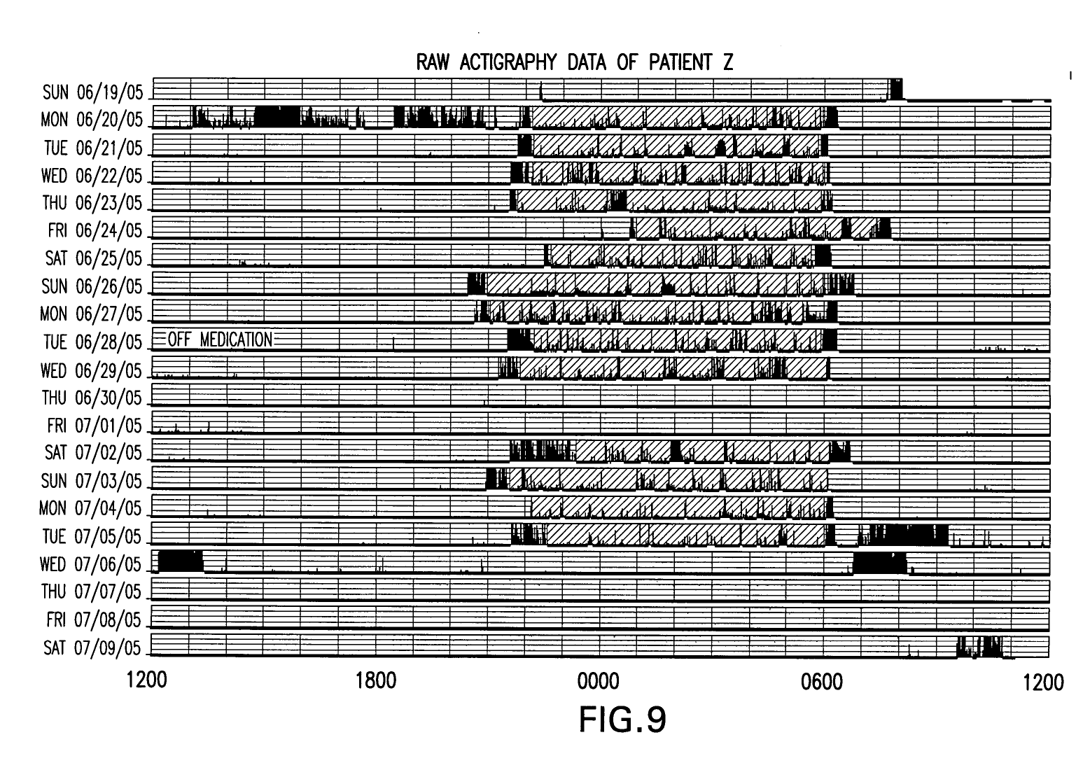

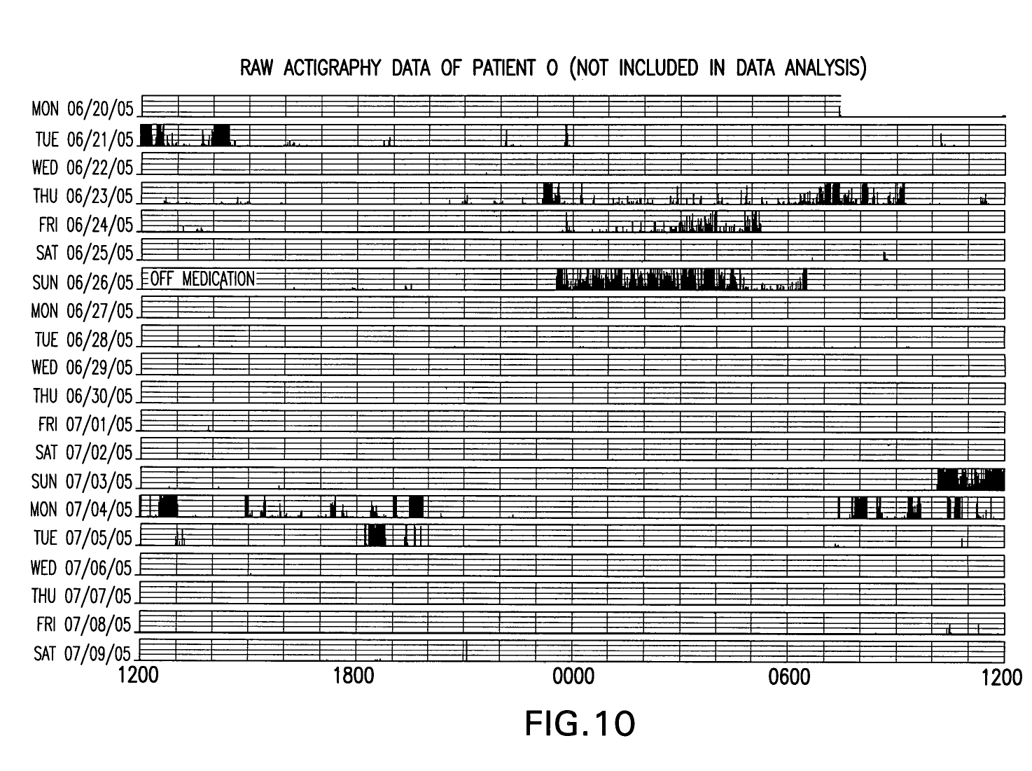

The raw data of each child are presented in FIGS. 4-10. The shaded (green) areas are those defined as the sleep period from sleep onset to morning rise time. The thin line (red) underneath the raw data reflects sleep-wake scoring. The line (in red) represents sleep and intermissions (white) represent wake minutes.

Comparison Between ON and OFF Medication Periods

To determine the effect of the sublingual tizanidine formulation on the Actigraphic sleep measures, the mean score of each sleep measure was calculated for each child over the days of each monitoring period (ON/OFF medication). ANOVA for repeated measures was conducted to test the effect on each individual measure. Table 12 summarizes the results.

TABLE 12
Actigraphic Sleep Measures During Periods
On and Off Medication (N = 6)

| | On Med. | | OFF | | | |
|---|---|---|---|---|---|---|
| Sleep Measure | Mean (STD) | | Mean (STD) | | F | P< |
| Sleep Duration | 501 | (41.7) | 485 | (54.1) | 5.30 | N.S. |
| Mean Activity Level | 12.40 | (8.89) | 17.27 | (8.54) | 7.23 | .05 |
| Wake Minute (N) | 53.83 | (49.4) | 83.49 | (53.6) | 16.57 | .01 |
| Sleep Minutes (N) | 447 | (56.1) | 402 | (57.7) | 29.72 | .005 |
| Sleep Efficiency (%) | 89.47 | (10.05) | 83.00 | (10.76) | 18.78 | .01 |
| Wake Episodes (N) | 13.44 | (9.03) | 18.26 | (7.75) | 15.01 | .05 |
| Longest Wake | 13.13 | (10.45) | 20.99 | (12.35) | 7.51 | .05 |
| Episode (min) | | | | | | |
| Sleep Episodes (N) | 14.41 | (9.03) | 19.23 | (7.79) | 15.44 | .05 |
| Longest Sleep | 190.4 | (110.6) | 124.1 | (59.0) | 4.00 | N.S |
| Episode (min) | | | | | | |

The results reflect significant improvement on most Actigraphic sleep measures. In comparison to the OFF medication period, the following differences were significant during the ON medication period:

Lower activity level during sleep

Shorter time of wakefulness during sleep

Longer time of sleep

Higher sleep efficiency

Smaller number of wake and sleep episodes (less sleep fragmentation)

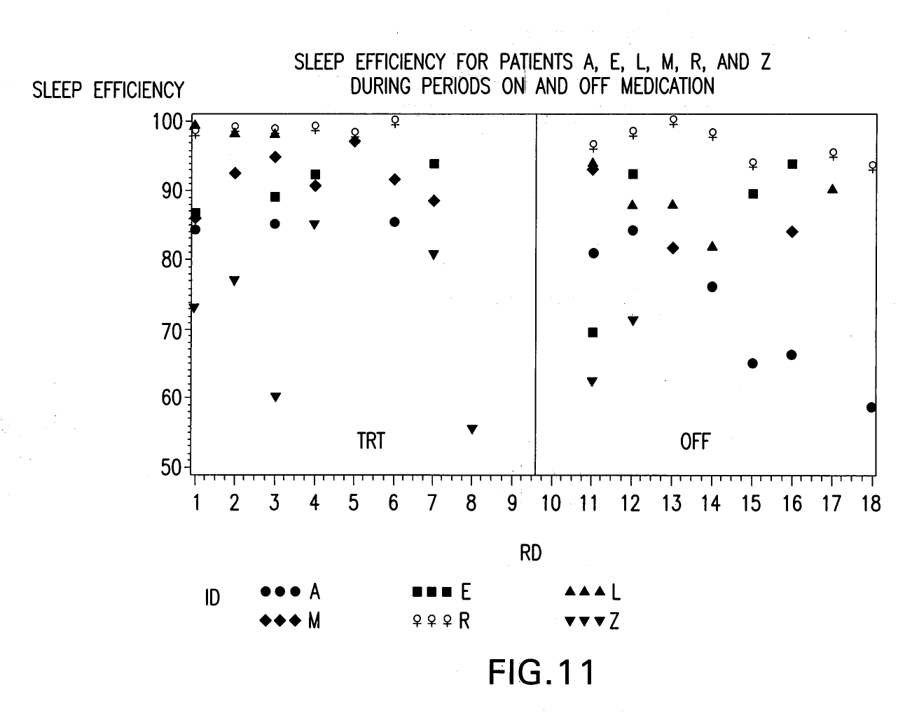

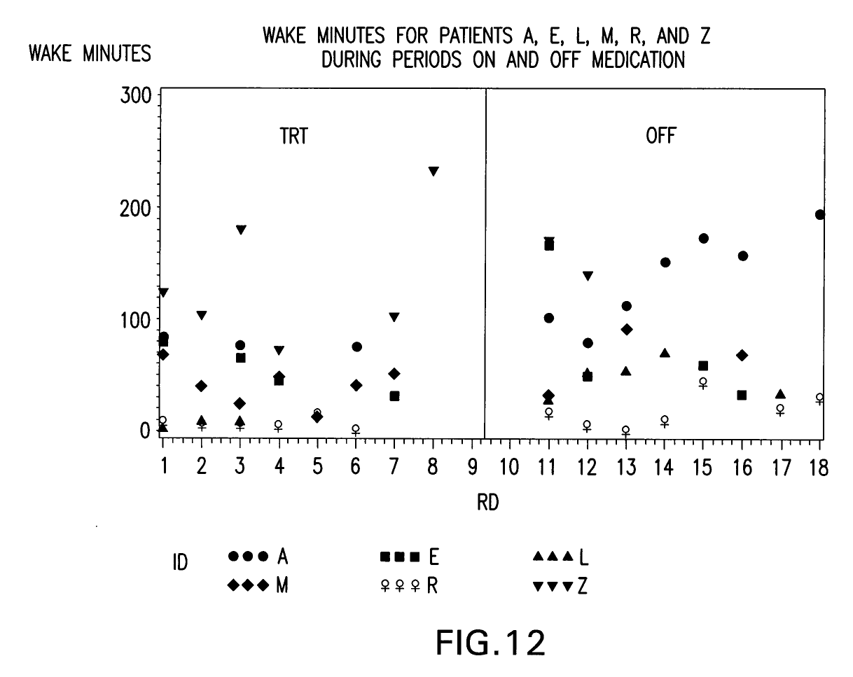

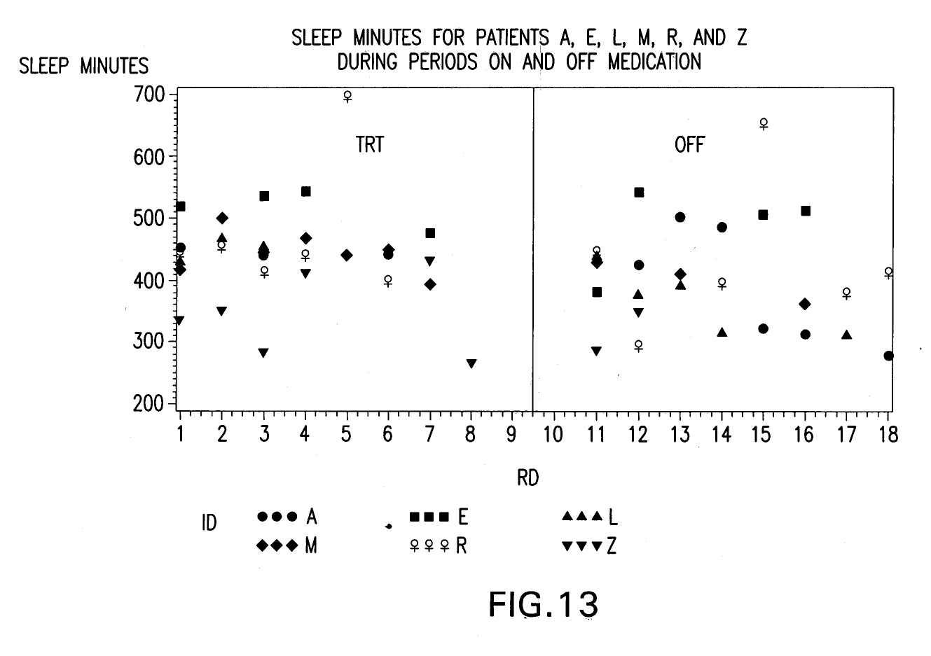

FIGS. 11-13 and Table 13 reflect these changes at the individual level for 6 subjects. RD relates to the running day of the study but for the OFF drug medication day 11 is day 1, day 12 is day 2 and so forth.

TABLE 13
Individual Changes in Actigraphic Sleep Measures

| | Sleep Period | | Mean Act Lev | | STD of Act Lev | | Wake Minutes | |
|---|---|---|---|---|---|---|---|---|
| Name | Off | Treat | Off | Treat | Off | Treat | Off | Treat |
| A | 534.14 | 525.00 | 26.45 | 15.21 | 57.16 | 41.12 | 139.00 | 79.00 |
| | (67.21) | (10.39) | (12.71) | (0.17) | (20.12) | (1.26) | (41.53) | (4.36) |
| E | 563.25 | 574.00 | 13.19 | 11.52 | 31.00 | 27.26 | 77.25 | 55.25 |
| | (20.41) | (44.36) | (5.53) | (1.85) | (6.63) | (0.96) | (60.80) | (21.61) |
| L | 415.40 | 458.00 | 13.77 | 4.47 | 31.80 | 13.87 | 47.60 | 6.00 |
| | (49.25) | (20.66) | (2.70) | (0.24) | (4.77) | (0.86) | (16.82) | (3.46) |
| M | 465.67 | 486.71 | 14.68 | 10.13 | 33.05 | 26.40 | 64.33 | 40.71 |
| | (35.23) | (33.26) | (4.57) | (1.91) | (8.62) | (4.40) | (30.27) | (18.03) |
| R | 457.57 | 479.50 | 6.71 | 4.60 | 18.55 | 14.25 | 17.29 | 5.67 |
| | (122.16) | (115.02) | (2.10) | (1.19) | (5.21) | (2.64) | (14.84) | (4.27) |
| Z | 474.50 | 484.00 | 28.81 | 28.45 | 59.32 | 55.12 | 155.50 | 136.33 |
| | (21.92) | (31.48) | (1.92) | (8.13) | (7.75) | (10.88) | (21.92) | (59.43) |
| | | | | | | | Mean Wake | |
| | Sleep Minutes | | Sleep Efficiency | | Wake Episodes | | Episode | |
| Name | Off | Treat | Off | Treat | Off | Treat | Off | Treat |
| A | 395.14 | 446.00 | 73.36 | 84.96 | 25.57 | 19.33 | 5.75 | 4.36 |
| | (89.02) | (6.08) | (9.81) | (0.53) | (6.63) | (6.81) | (2.21) | (1.20) |
| E | 486.00 | 518.75 | 86.40 | 90.52 | 27.75 | 24.75 | 2.58 | 2.21 |
| | (70.25) | (29.42) | (11.32) | (3.24) | (14.93) | (8.26) | (0.58) | (0.31) |
| L | 367.80 | 452.00 | 88.37 | 98.71 | 13.00 | 3.33 | 4.20 | 3.38 |
| | (54.42) | (17.35) | (4.41) | (0.72) | (6.52) | (3.21) | (2.18) | (4.00) |
| M | 401.33 | 446.00 | 86.28 | 91.69 | 13.00 | 11.71 | 4.89 | 3.40 |
| | (33.56) | (33.60) | (5.99) | (3.78) | (1.00) | (3.90) | (2.21) | (0.64) |
| R | 440.29 | 473.83 | 96.51 | 98.91 | 8.71 | 2.83 | 1.62 | 3.04 |
| | (112.62) | (111.17) | (2.49) | (0.63) | (7.25) | (1.94) | (0.84) | (4.91) |
| Z | 319.00 | 347.67 | 67.09 | 72.04 | 21.50 | 18.67 | 7.54 | 7.17 |
| | (43.84) | (67.24) | (6.14) | (11.67) | (7.78) | (5.20) | (1.71) | (1.77) |

| | Long Wake | | Longest Wake | | | | Mean Sleep | |
|---|---|---|---|---|---|---|---|---|
| | Episodes | | Episode | | Sleep Episodes | | Episode | |
| Name | Off | Treat | Off | Treat | Off | Treat | Off | Treat |
| A | 8.14 | 6.00 | 33.29 | 15.00 | 26.57 | 20.33 | 15.39 | 23.42 |
| | (1.46) | (1.00) | (23.86) | (2.65) | (6.63) | (6.81) | (4.07) | (6.72) |
| E | 4.25 | 1.50 | 15.50 | 13.50 | 28.75 | 25.75 | 20.95 | 21.75 |
| | (3.20) | (0.58) | (10.41) | (6.19) | (14.93) | (8.26) | (10.85) | (6.69) |
| L | 2.80 | 0.33 | 18.00 | 3.67 | 14.00 | 4.33 | 30.12 | 143.57 |
| | (1.10) | (0.58) | (11.34) | (3.79) | (6.52) | (3.21) | (12.31) | (84.81) |
| M | 3.67 | 2.86 | 17.67 | 11.14 | 14.00 | 12.57 | 28.88 | 43.87 |
| | (1.53) | (1.77) | (4.93) | (5.81) | (1.00) | (4.24) | (4.39) | (30.09) |
| R | 0.71 | 0.17 | 4.00 | 3.50 | 9.57 | 3.83 | 110.99 | 183.90 |
| | (0.95) | (0.41) | (2.65) | (4.72) | (7.32) | (1.94) | (173.41) | (147.99) |
| Z | 8.50 | 9.67 | 37.50 | 32.00 | 22.50 | 19.67 | 15.44 | 19.17 |
| | (4.95) | (3.39) | (20.51) | (12.33) | (7.78) | (5.20) | (7.28) | (7.30) |

| | |
|---|---|
| | Longest Sleep |
| Long Sleep Episode | Episode |

| Name | Off | Treat | Off | Treat |
|---|---|---|---|---|
| A | 13.86 | 13.00 | 72.00 | 80.67 |
| | (4.06) | (3.61) | (17.86) | (30.01) |
| E | 13.75 | 11.75 | 165.50 | 158.25 |
| | (3.69) | (3.50) | (101.33) | (90.43) |
| L | 9.60 | 3.00 | 122.40 | 335.33 |
| | (3.91) | (1.73) | (38.10) | (21.13) |
| M | 8.33 | 7.43 | 126.67 | 180.71 |
| | (0.58) | (1.72) | (32.88) | (76.85) |
| R | 6.14 | 2.83 | 209.43 | 309.83 |
| | (4.85) | (1.17) | (134.56) | (81.01) |
| Z | 14.50 | 12.17 | 48.50 | 77.50 |
| | (3.54) | (2.40) | (16.26) | (15.80) |

Analysis by Quarters of the Night

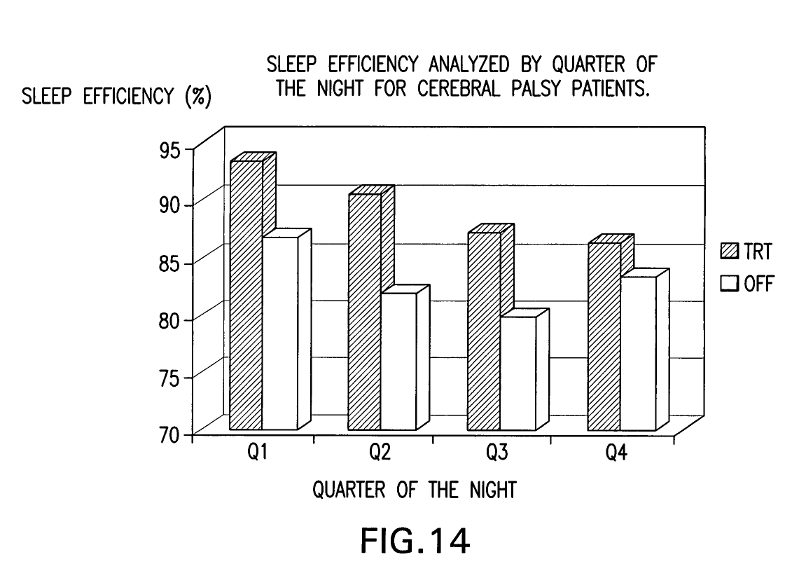

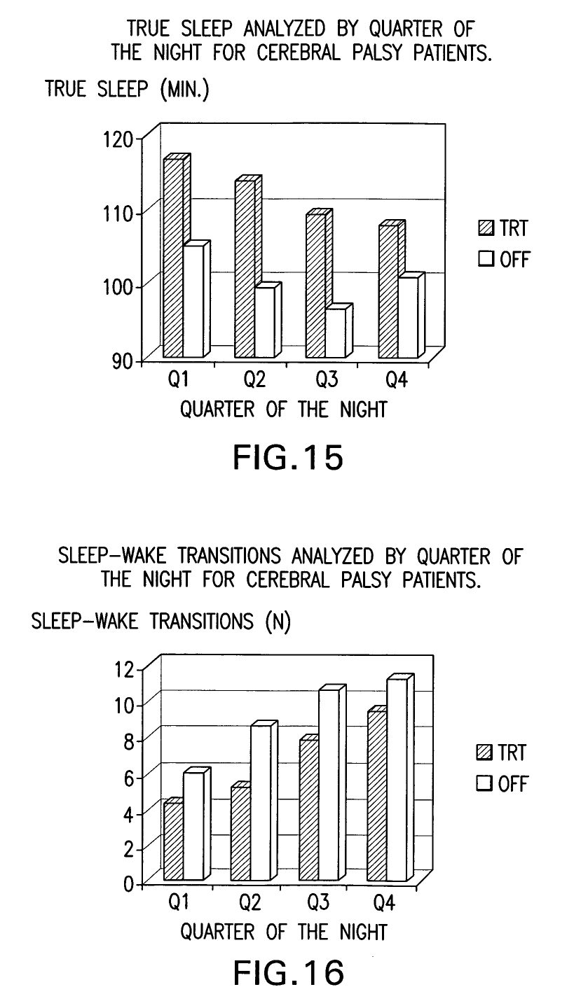

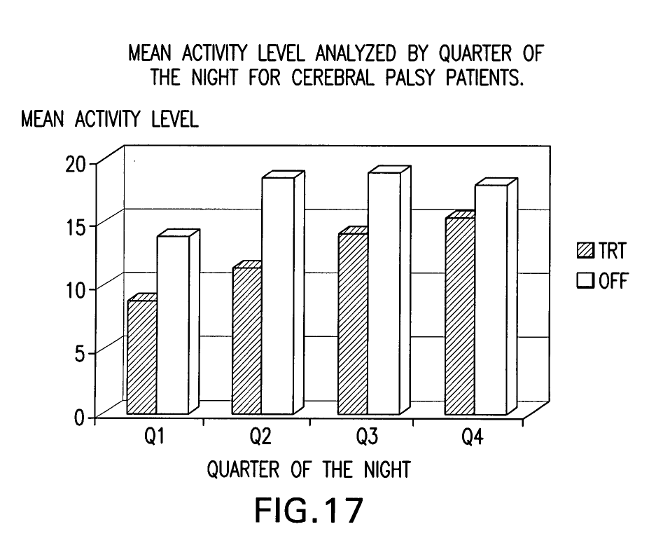

To examine the possibility that the medication effects had a specific time course during the night, each night was divided to 4 equal quarters and the analyses were repeated with quarter of the night as an additional factor. FIGS. 14-17 summarize the results. For all the measures in the figures, significant main effects were found for condition (TRT vs. OFF). Significant effects for Quarter of the night were found for mean activity level during the sleep period. However, no significant effects for Quarter interaction was found for any of the sleep quality measures. The quarter effect reflects the well-documented normal sleep phenomenon by which most of the deep and quiet sleep is concentrated in the first parts of the night, while the later parts of the night are characterized by more active and fragmented sleep.

Discussion and Conclusions

The results of this study suggested that children with CP sleep significantly better while they receive a sublingual tizanidine formulation. Using the sleep-wake scoring algorithm as used in normal children the actigraphic measures indicated that on medications these children slept significantly longer, had higher sleep efficiency, and reduced number of waking periods and sleep-wake transitions.

The analysis for testing the course of the effect during the night revealed no specific time course for the effect of medication. Therefore the data suggests that the effect was continuous through most of the night with a slight tendency for the effect to be somewhat smaller in the last quarter of the night (see FIGS. 14-17). This tendency might be related to the fact that the last quarter of the night was usually the most fragmented and shallow in normal sleep.

### Example 5

### Treatment of Spasticity in Patients with Multiple Sclerosis by pre-Bedtime Administration of Sublingual or Oral Tizanidine

Twenty patients suffering from spasticity as a complication of multiple sclerosis (MS) were treated with placebo, oral tizanidine (8 mg) and sublingual tizanidine (8 mg) in a double blind, double dummy, crossover study to determine the effect of tizanidine dosed once nightly before bedtime on next day spasticity and to investigate the level of somnolence occurring the next day. Spasticity was studied using the Ashworth scale and somnolence was studied by measuring the Epworth Sleepiness scale (ESS). The Ashworth scale was measured by an examiner with experience in its use, while the Epworth scale was measured from a validated questionnaire filled out by the patients. Data was considered comparable only if all spasticity measurement sessions of a particular patient were carried out by the same doctor. The patients were further evaluated by actigraphy to measure the quality and extent of their sleep. The sleep data was analyzed by night quarters to see the effect on the different quarters of the night time sleep.

Patients were selected based upon various criteria. Patients had to be within an age of 20 and 65, diagnosed for MS, have an EDSS≦6.5 at screening and have spasticity requiring treatment. Females who participated in the study had to agree to use a medically accepted form of birth control, be surgically sterile, or be two years post-menopausal. Oral contraception was not acceptable as this was contraindicated for tizanidine use.

Patients were excluded if they met any of the following criteria.
1. An acute multiple sclerosis exacerbation requiring the treatment of steroids within 30 days of screening.
2. Previous diagnosis of a sleep disorder distinct from MS, such as obstructive sleep apnea or narcolepsy.
3. Previous history of dementia, unstable psychiatric disease, neurological or medical disorder, fibromyalgia, or chronic pain (not related to MS).
4. At screening having a score>18 on the Beck Depression Inventory indicating that the patient had more than a mild case of depression.
5. Currently taking a tizanidine HCl dose higher than 8 mg (per dose) for treatment.
6. Changes in chronic oral medications within 2 weeks of screening.
7. The initiation or discontinuation of interferon beta within 30 days of screening.
8. Use of a Baclofen pump.
9. Taking medications that would potentially interfere with the actions of the study medication or outcome variables such as anti-hypertensives, hypnotics, tranquilizers, antihistamines (except non-sedating), 4-amino pyridine, anticonvulsants, amphetamines benzodiazepines, tricyclic antidepressants, clonidine, Ritalin, modafanil, amantadine or other stimulants within 2 weeks or 5 half-lives of the start of the study.
10. Use of CYP1A2 inhibitors (e.g. ciprofloxacin or fluvoxamine) for the duration of the study.
11. Significant abnormalities in clinical screening laboratory parameters as described below:
-ALT >3×ULN -AST >3×ULN -Creatinine >2.0 mg/dL
-Bilirubin >3×ULN -WBC <2,300/mm³ -Platelets <80,000/mm³
-Systolic BP <90 mmHg or symptomatic hypotension
-ECG e.g. clinically significant arrhythmias or recent history of myocardial infarction
12. Within the last 30 days prior to study start, worked a rotating or night shift schedule.
13. Have a history of allergy to tizanidine or any inactive component (including lactose intolerance) of either the test or reference formulations.
14. History of substance abuse within past 12 months.
15. Participation in another clinical trial within 30 days of study start.
16. Patients who are non-cooperative or unwilling to sign consent form.

The study was designed to be a double-blind, double-dummy randomized, three-treatment, two-way crossover, comparative, placebo-controlled clinical efficacy and safety sleep-study. Prior to the study, all patients received a placebo once nightly for 7 days (phase 1). Thereafter, patients were placed into one of 2 groups. The first group, Group A, received oral tizanidine HCl in an 8 mg dose once nightly for 7 days (phase 2). Following this oral phase, the patients received 8 mg sublingual tizanidine nightly for an additional 7 days (phase 3). The second group, Group B, received sublingual tizanidine HCl 8 mg once nightly for 7 days (phase 2). Thereafter, the patients received 8 mg oral tizanidine nightly for an additional 7 days (phase 3). Table 14 illustrates the scheduled visits for each group.

TABLE 14
Group A and Group B Schedule

| | | Medication Schedule | | | |
|---|---|---|---|---|---|
| Phase | Visit | Group A | Group B | Administration | Study Days |
| | 1 | — | | | Within 30 |
| | 2 | Dispensing | Dispensing | | 0 |
| 1 | 3 | Placebo | Placebo | 1 | 1-7 |
| 2 | 4 | Oral | Sublingual | 2 | 8-14 |
| 3 | 5 | Sublingual | Oral | 3 | 15-21 |

At each nightly dosing, the patients were required to take both a sublingual dose and an oral dose wherein one dose had the active drug and the other had a placebo. The doses for the tizanidine or placebo were administered either sublingually (1 tablet) or orally (2 tablets). Two tablets were necessary for the oral dose because the commercially available oral tizanidine is only manufactured as a 4 mg tablet; however, the sublingual test tablet was available as an 8 mg tizanidine HCl tablet.

The dose administration followed three different phases. The first phase, Placebo Reference, consisted of administering a sublingual placebo and two oral placebo tablets. The second phase, Oral Tizanidine Reference, consisted of administering two oral tizanidine HCl tablets (4 mg each) and one sublingual placebo tablet. The third phase, Sublingual Tizanidine Test, consisted of administering one sublingual tizanidine HCl tablet (8 mg) and two oral placebo tablets. In all phases, the patients were required to take 3 tablets even when patients were assigned the active drug.

The Ashworth scale scores were evaluated during the clinic visits (as measured the next day at 11 AM or later) at the end of each phase. The Epworth Sleepiness Scale (ESS) questionnaire was filled out during the visit. Patients were monitored for all 3 phases by nightly actigraphy and a home sleep-diary was kept during the duration of the trial. Efficacy parameters were based upon the prior evaluation criteria and subjective measures of sleep.

The Results of the next day spasticity were determined based upon Ashworth score evaluations. Table 15 summarizes the scores for all patients for each visit.

TABLE 15

| | | | Left leg | Right leg | Total |
|---|---|---|---|---|---|
| Patient | | | total | total | Ashworth |
| Number | Visit number | Examiner | for limb | for limb | Score |
| 1 | 0 = screen | V V | 2 | 2 | 4 |
| | 3 = placebo | V V | 2 | 2 | 4 |
| | 4 = SL* | V V | 2 | 2 | 4 |
| | 5 = oral | V V | 1 | 2 | 3 |
| 2 | 0 = screen | V V | 8 | 9 | 17 |
| | 3 = placebo | V V | 8 | 8 | 16 |
| | 4 = oral | V V | 7 | 6 | 13 |
| | 5 = SL | V V | 6 | 7 | 13 |
| 3 | 0 = screen | V V | 4 | 9 | 18 |
| | 3 = placebo | V V | 9 | 9 | 18 |
| | 4 = SL | V V | 9 | 9 | 18 |
| | 5 = oral | V V | 9 | 9 | 18 |
| 4 | 0 = screen | A-K | 5 | 5 | 10 |
| | 3 = placebo | A-K | 4 | 3 | 7 |
| | 4 = SL | A-K | 1 | 2 | 3 |
| | 5 = oral | A-K | 3 | 2 | 5 |
| 5 | 0 = screen | A-K | 0 | 3 | 3 |
| | 3 = placebo | A-K | 2 | 3 | 5 |
| | 4 = oral | A-K | 0 | 2 | 2 |
| | 5 = SL | A-K | 0 | 1 | 1 |
| 6 | 0 = screen | V V | 9 | 9 | 18 |
| | 3 = placebo | V V | 9 | 9 | 18 |
| | 4 = oral | V V | 8 | 9 | 17 |
| | 5 = SL | V V | 8 | 7 | 15 |
| 7 | 0 = screen | A-K | 3 | 2 | 5 |
| | 3 = placebo | A-K | 2 | 2 | 4 |
| | 4 = SL | A-K | 1 | 1 | 2 |
| | 5 = oral | A-K | 1 | 1 | 2 |
| 8 | 0 = screen | V V | 9 | 9 | 18 |
| | 3 = placebo | V V | 9 | 9 | 18 |
| | 4 = oral | V V | 9 | 9 | 18 |
| | 5 = SL | V V | 8 | 8 | 16 |
| 9 | 0 = screen | V V | 8 | 7 | 15 |
| | 3 = placebo | V V | 7 | 7 | 14 |
| | 4 = SL | V V | 6 | 6 | 12 |
| | 5 = oral | V V | 6 | 6 | 12 |
| 10 | 0 = screen | V V | 3 | 5 | 8 |
| | 3 = placebo | V V | 3 | 5 | 8 |
| | 4 = SL | V V | 3 | 4 | 7 |
| | 5 = oral | V V | 3 | 3 | 6 |
| 11 reject - | 0 = screen | V V | 3 | 4 | 7 |
| incomplete | 3 = placebo | V V | 3 | 3 | 6 |
| | 5 = oral | V V | 3 | 4 | 7 |
| 12 | 0 = screen | V V | 6 | 4 | 10 |
| | 3 = placebo | V V | 6 | 4 | 10 |
| | 4 = oral | V V | 5 | 3 | 8 |
| | 5 = SL | V V | 5 | 3 | 8 |
| 13 reject - | 0 = screen | V V | 8 | 6 | 14 |
| incomplete | 3 = placebo | V V | 8 | 6 | 14 |
| 14 reject - | 0 = screen | V V | 3 | 6 | 9 |
| different | 3 = placebo | V V | 5 | 4 | 9 |
| examiners | 4 = oral | A-K | 0 | 1 | 1 |
| | 5 = SL | V V | 2 | 3 | 5 |
| 15 | 0 = screen | V V | 5 | 4 | 9 |
| | 3 = placebo | V V | 4 | 4 | 8 |
| | 4 = oral | V V | 4 | 5 | 8 |
| | 5 = SL | V V | 1 | 2 | 3 |
| 16 | 0 = screen | V V | 9 | 9 | 18 |
| | 3 = placebo | V V | 9 | 9 | 18 |
| | 4 = oral | V V | 5 | 6 | 11 |
| | 5 = SL | V V | 4 | 4 | 8 |
| 17 | 0 = screen | V V | 6 | 6 | 12 |
| | 3 = placebo | V V | 5 | 5 | 10 |
| | 4 = SL | M-L | 2 | 1 | 3 |
| | 5 = oral | V V | 3 | 3 | 6 |
| 18 reject - | 0 = screen | V V | 6 | 6 | 12 |
| incomplete | | | | | |
| 19 | 0 = screen | V V | 4 | 6 | 10 |
| | 3 = placebo | V V | 6 | 6 | 12 |
| | 4 = SL | V V | 7 | 6 | 13 |
| | 5 = oral | V V | 6 | 7 | 13 |
| 21 | 0 = screen | V V | 8 | 6 | 14 |
| | 3 = placebo | V V | 6 | 7 | 13 |
| | 4 = SL | V V | 2 | 1 | 3 |
| | 5 = oral | V V | 5 | 4 | 9 |

*SL = sublingual

Table 16 summarizes the statistical values calculated for the left limb, the right limb, and the overall Ashworth score. The data within the table demonstrated that the mean and median values improved as the treatment progressed from placebo to oral treatment to sublingual treatment.

TABLE 16
Statistical Values for the Left Limb

| Total for Left Limb | N | Mean | Std | Minimum | Median | Maximum |
|---|---|---|---|---|---|---|
| Placebo | 17 | 5.6 | 2.6 | 2.0 | 6.0 | 9.0 |
| Oral | 17 | 4.4 | 2.9 | 0.0 | 5.0 | 9.0 |
| Sublingual | 17 | 3.9 | 2.9 | 0.0 | 3.0 | 9.0 |

Statistical Values for the Right Limb

| Total for Right Limb | N | Mean | Std | Minimum | Median | Maximum |
|---|---|---|---|---|---|---|
| Placebo | 17 | 5.6 | 2.5 | 2.0 | 5.0 | 9.0 |
| Oral | 17 | 4.6 | 2.8 | 1.0 | 4.0 | 9.0 |
| Sublingual | 17 | 3.9 | 2.7 | 1.0 | 3.0 | 9.0 |

Statistical Values for Total Ashworth Score

| Total Ashworth Score | N | Mean | Std | Minimum | Median | Maximum |
|---|---|---|---|---|---|---|
| Placebo | 17 | 11.3 | 5.0 | 4.0 | 10.0 | 18.0 |
| Oral | 17 | 8.9 | 5.6 | 1.0 | 8.0 | 18.0 |
| Sublingual | 17 | 7.9 | 5.5 | 1.0 | 7.0 | 18.0 |

As illustrated in Table 16, the overall Ashworth score for the mean improved from 11.3 to 8.9 as treatment progressed from placebo to oral treatment and from 11.3 to 7.9 as treatment progressed from oral treatment to sublingual treatment. The median values paralleled the mean with the improvement being from 10 to 8 and 10 to 7, respectively. Table 17, below, summarizes the statistical significance of these measured differences between the placebo treatment and the two test treatments. The improvement in spasticity scores for the oral vs. placebo treatments was significant (defined as P<0.05 ) for each limb (as separately tested) and highly significant (P<0.001) for the overall Ashworth score. For the sublingual vs. placebo treatments the improvements were highly significant for each limb as separately tested and for the overall Ashworth score.

TABLE 17
Statistical data for the Ashworth Test as determined from the Placebo Treatment

| Total | | | | | | | | | | | | | | |
|---|---|---|---|---|---|---|---|---|---|---|---|---|---|---|
| Ashworth | Oral-Placebo | | | | | | | Sublingual-Placebo | | | | | | |
| Scores | N | Mean | Std | Min | Median | Max | P | N | Mean | Std | Min | Median | Max | P |
| Total Left | 17 | −1.24 | 1.39 | −5.00 | −1.00 | 0.00 | 0.002 | 17 | −1.71 | 1.61 | −5.00 | −1.00 | 1.00 | <.001 |
| Limb | | | | | | | | | | | | | | |
| Total | 17 | −1.06 | 1.30 | −3.00 | −1.00 | 1.00 | 0.004 | 17 | −1.71 | 1.72 | −6.00 | −1.00 | 0.00 | <.001 |
| Right | | | | | | | | | | | | | | |
| Total | 17 | −2.35 | 2.40 | −8.00 | −2.00 | 1.00 | <.001 | 17 | −3.41 | 3.16 | −10.00 | −3.00 | 1.00 | <.001 |

The overall Ashworth score results of the evaluated patients (those patients who were evaluated by a single examiner and who completed all arm tests) were compared for the sublingual treatment and oral treatment. Table 18 summarizes the results of the comparison and their difference

TABLE 18
Comparison of the Total Sublingual and Oral Ashworth Scores

| Patient Number | Total Sublingual | Total Oral | Difference |
|---|---|---|---|
| 1 | 4 | 3 | 1 |
| 2 | 13 | 13 | 0 |
| 3 | 18 | 18 | 0 |
| 4 | 3 | 5 | −2 |
| 5 | 1 | 2 | −1 |
| 6 | 15 | 17 | −2 |
| 7 | 2 | 2 | 0 |
| 8 | 16 | 18 | −2 |
| 9 | 12 | 12 | 0 |
| 10 | 7 | 6 | 1 |
| 12 | 8 | 8 | 0 |
| 15 | 3 | 8 | −5 |
| 16 | 8 | 11 | −3 |
| 19 | 13 | 13 | 0 |
| 21 | 3 | 9 | −6 |

the statistical calculations (mean and median) showed that there was greater improvement in next day spasticity in the sublingual treatment when compared to the oral treatment that this improvement was statistically significant (p=0.034). See Table 19.

TABLE 19
Statistical Comparison of Sublingual and Oral Ashworth Scores

| Total | | | | | | | |
|---|---|---|---|---|---|---|---|
| Ashworth | | | | | | | |
| Scores | N | Mean | Std | Minimum | Median | Maximum | P |
| Total | 15 | 8.40 | 5.69 | 1.00 | 8.00 | 18.00 | |
| Ashworth | | | | | | | |
| Sublingual | | | | | | | |
| Total | 15 | 9.67 | 5.51 | 2.00 | 9.00 | 18.00 | |
| Ashworth | | | | | | | |
| Oral | | | | | | | |
| Changes | 15 | −1.27 | 2.09 | −6.00 | 0.00 | 1.00 | 0.034 |
| (Sublingual- | | | | | | | |
| Oral) | | | | | | | |

Results—Next Day Somnolence Table 20 summarizes the average results of the Epworth Sleepiness scale for the patients in the trial. Despite the known high prevalence of somnolence as an adverse effect of daytime tizanidine treatment, it was expected that somnolence during treatment would be comparable to that found during placebo administration because the tizanidine was dosed before bedtime and the spasticity and ESS evaluations were done the next day after 11 AM. The mean values of the ESS demonstrated that sublingual treatment was superior to placebo treatment. Similarly, the statistical analysis demonstrated that the ESS values for oral treatment were not significantly better than those of placebo (P=0.303); however, the values for sublingual treatment were better than those of placebo (P=0.005).

TABLE 20
Statistical Results for Next Day Somnolence

| Phase | N | Mean | Std | Min | Median | Max |
|---|---|---|---|---|---|---|
| screening | 17 | 7.8 | 5.3 | 0 | 9 | 16 |
| placebo | 17 | 6.2 | 5.5 | 0 | 5 | 16 |
| oral | 17 | 5.2 | 4.6 | 0 | 6 | 13 |
| sublingual | 17 | 4.6 | 4.5 | 0 | 2 | 13 |

Results—Actigraphy

Table 21 below summarizes the average results of the various sleep parameters measured by actigraphy for the evaluated patients (N=14). These results were based on an entire night's data. There were improvements in many of the parameters for the two treatment groups when compared to placebo (sleep period, wake minutes, sleep minutes, sleep efficiency, mean and long wake episodes, long sleep episodes and longest sleep episodes).

TABLE 21
Actigraphy Results

| | Treatment | | | | | |
|---|---|---|---|---|---|---|
| | Oral | | Placebo | | Sublingual | |
| | Mean | Std | Mean | Std | Mean | Std |
| Sleep Period | 427.91 | 96.67 | 406.25 | 100.53 | 422.89 | 78.30 |
| Mean Act Lev | 13.72 | 5.03 | 14.67 | 6.36 | 13.20 | 4.53 |
| STD of Act Lev | 28.81 | 13.59 | 29.13 | 8.92 | 29.20 | 7.62 |
| Wake Minutes | 40.27 | 27.54 | 42.40 | 33.05 | 37.29 | 22.44 |
| Sleep Minutes | 387.64 | 87.79 | 363.84 | 100.33 | 385.60 | 78.53 |
| Sleep Efficiency | 91.01 | 5.53 | 89.60 | 7.42 | 91.26 | 4.96 |
| Wake Episodes | 7.23 | 4.32 | 6.45 | 3.72 | 6.91 | 3.86 |
| Mean Wake | 4.85 | 2.65 | 7.09 | 5.11 | 5.86 | 2.63 |
| Episode | | | | | | |
| Long Wake | 2.30 | 1.80 | 2.45 | 1.69 | 2.26 | 1.23 |
| Episodes | | | | | | |
| Longest Wake | 16.58 | 13.11 | 17.98 | 13.56 | 17.34 | 9.34 |
| Episode | | | | | | |
| Sleep Episodes | 8.21 | 4.33 | 7.39 | 3.75 | 7.91 | 3.86 |
| Mean Sleep | 87.57 | 65.72 | 76.02 | 38.48 | 67.48 | 33.96 |
| Episode | | | | | | |
| Long Sleep | 5.45 | 2.67 | 5.13 | 2.20 | 5.28 | 2.05 |
| Episodes | | | | | | |
| Longest Sleep | 183.13 | 67.64 | 174.14 | 63.77 | 177.22 | 64.27 |
| Episode | | | | | | |

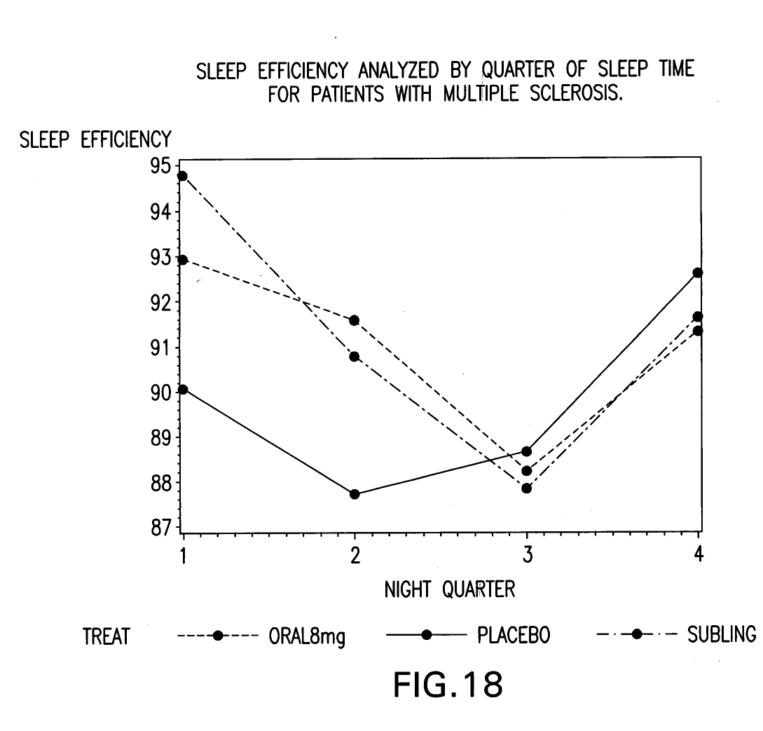

The actigraphy results were analyzed by dividing each night into four sleep periods. Overall, sleep efficiency below 90% suggests inadequate restful sleep. And restful sleep in the first half of the night has a significant effect on the usefulness of overall sleep. FIGS. 18-20 summarize the results of the quarterly night analysis. In the first quarter of the night, the oral and sublingual treatment improved sleep efficiency when compared to placebo treatment. In this case, sublingual treatment gave the larger improvement. See FIG. 18. This improved sleep efficiency continued for both the oral and sublingual treatments into the second quarter; however, the placebo, oral, and sublingual treatments became essentially equivalent after the second half of the sleep period. The overall effect was observed by the small differences when a full night sleep was evaluated.

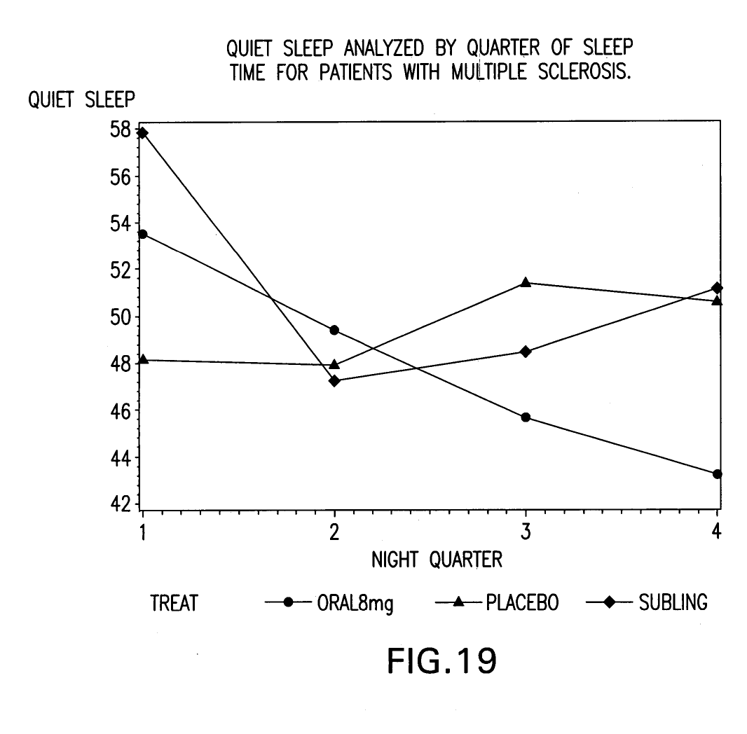

The trends observed in the quarterly sleep analysis were also observed in the “Quiet Sleep” analysis. FIG. 19 summarizes the “Quiet Sleep” results. Again, patients who in the sublingual treatment group had more “quiet sleep” in the first quarter of the night than those patients in the oral treatment group. In turn, patients in the oral treatment group had more “quiet sleep” than those in the placebo treatment group.

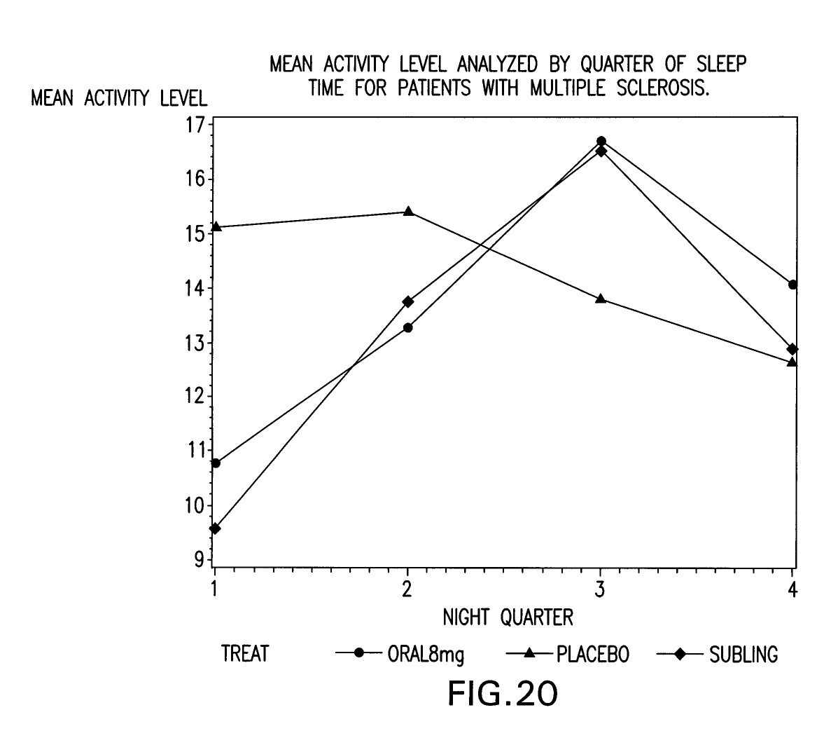

FIG. 20 summarizes the results for “Mean Activity Level.” In the first quarter, those patients in the placebo treatment group showed a considerably higher level of “mean activity” during sleep than those patients in the sublingual or oral treatment group. The sublingual treatment gave a somewhat improved “mean activity level” value than oral treatment. Again as before, the differences existing for each treatment during the first quarter of sleep, were lessened during the second half of sleep. Nevertheless, based on the actigraphy studies, one can conclude that the sublingual tizanidine improved the quality of sleep in the first and second quarters of sleep. This may explain the improved ESS scores for patients in the sublingual treatment.

Thus, the study demonstrated that delivery of tizanidine before bedtime improved the spasticity of MS patients, where sublingual tizanidine HCl administration gave statistically significant greater improvements. The fact that improvements were based upon a full score in the Ashworth scale was clinically significant. Also, next day spasticity improved without the adverse effect of somnolence. As illustrated above, ESS scores for patients who received tizanidine HCl sublingually had less somnolence than those patients receiving placebo. Consequently, there can be no somnolence attributed to the sublingual tizanidine treatment. It is believed that the decrease in somnolence may be attributed to improved sleep, as determined by the actigraphy data.

1-73. (canceled)

74. A method of fabricating integrated circuitry comprising:
forming an aluminum-comprising conductive metal line over a semiconductor substrate; and
exposing at least sidewalls of the aluminum-comprising conductive metal line to a solution comprising an inorganic acid, hydrogen peroxide and a carboxylic acid buffering agent selected from the group consisting of ammonium citrate, ammonium oxalate, and mixtures thereof.

75. The method of claim 74 wherein the carboxylic acid buffering agent comprises ammonium citrate.

76. The method of claim 75 wherein the ammonium citrate is derived at least in part from adding diammonium citrate salt to an aqueous solution.

77. The method of claim 74 wherein the carboxylic acid buffering agent comprises ammonium oxalate.

78. The method of claim 74 wherein the carboxylic acid buffering agent comprises ammonium citrate and ammonium oxalate.

79. The method of claim 74 wherein the inorganic acid comprises H₃PO₄.

80. The method of claim 74 wherein the inorganic acid comprises H₂SO₄.

81. The method of claim 74 wherein the solution has a pH of from 3.8-4.8.

82. The method of claim 74 wherein the conductive metal line consists essentially of metal.

83. A method of fabricating integrated circuitry comprising:
forming a patterned carbon-containing masking layer over an aluminum-comprising layer;
using the masking layer, etching the aluminum-comprising layer to form an aluminum-comprising conductive metal line;
after forming the aluminum-comprising conductive metal line, etching the carbon-containing masking layer from the substrate, at least one of the carbon-containing masking layer etching and the aluminum-comprising layer etching leaving a polymer residue over at least some of the aluminum-comprising conductive metal line;
providing an immersion bath containing a solution comprising sulfuric acid, hydrogen peroxide, and a carboxylic acid buffering agent selected from the group consisting of ammonium citrate, ammonium oxalate, and mixtures thereof; the sulfuric acid being present in the bath at from 1.0 weight percent to 15.0 weight percent, the hydrogen peroxide being present in the bath at from 1.0 weight percent to 15.0 weight percent, the bath having a pH from 3.0 to 7.0; and
after etching the carbon-containing masking layer, immersing the substrate within the bath under conditions effective to etch at least some of the polymer residue from over the aluminum-comprising conductive metal line.

84. The method of claim 83 wherein the carbon-containing masking layer comprises photoresist.

85. The method of claim 83 wherein the bath has a pH of from 3.8-4.8.

86. The method of claim 83 wherein the carboxylic acid buffering agent comprises ammonium citrate.

87. The method of claim 83 wherein the ammonium citrate is derived at least in part from adding diammonium citrate salt to an aqueous solution.

88. The method of claim 83 wherein the carboxylic acid buffering agent comprises ammonium oxalate.

89. The method of claim 83 wherein the carboxylic acid buffering agent comprises ammonium citrate and ammonium oxalate.

90. The method of claim 83 wherein the carbon-containing masking layer etching leaves a polymer residue over at least some of the aluminum-comprising conductive metal line.

91. The method of claim 83 wherein the aluminum-comprising layer etching leaves a polymer residue over at least some of the aluminum-comprising conductive metal line
